SEQUENCE LISTING (475 sequences)

SEQ ID NO: 1 - protein, 9 aa, Artificial Sequence
  Synthetic Peptide
  MISC_FEATURE (2)..(2): Xaa = Any amino acid
  MISC_FEATURE (4)..(4): Xaa = Serine or Threonine
  MISC_FEATURE (7)..(7): Xaa = Tyrosine or Phenylalanine
CXDXFVXQC

SEQ ID NO: 2 - protein, 17 aa, Artificial Sequence
  Synthetic Peptide
MDVRCRDSFVYQCHVGT

SEQ ID NO: 3 - protein, 8 aa, Artificial Sequence
  Synthetic Peptide
FPEQPFPE

SEQ ID NO: 4 - protein, 8 aa, Artificial Sequence
  Synthetic Peptide
QPEQAFPE

SEQ ID NO: 5 - protein, 12 aa, Artificial Sequence
  Synthetic Peptide
  MISC_FEATURE (2)..(2): Xaa = Proline, Arginine, or Methionine
PXEPQPEQPFPE

SEQ ID NO: 6 - protein, 12 aa, Artificial Sequence
  Synthetic Peptide
PPEPQPEQAFPE

SEQ ID NO: 7 - protein, 12 aa, Artificial Sequence
  Synthetic Peptide
PREPQPEQAFPE

SEQ ID NO: 8 - protein, 12 aa, Artificial Sequence
  Synthetic Peptide
PMEPQPEQPFPE

SEQ ID NO: 9 - protein, 15 aa, Artificial Sequence
  Synthetic Peptide
RGRAQPEQAFPESVG

SEQ ID NO: 10 - protein, 11 aa, Artificial Sequence
  Synthetic Peptide
VVKGGSSSLGW

SEQ ID NO: 11 - protein, 6 aa, Artificial Sequence
  Synthetic Peptide
  MISC_FEATURE (2)..(2): Xaa = Any amino acid
DXFVYQ

SEQ ID NO: 12 - protein, 6 aa, Artificial Sequence
  Synthetic Peptide
  MISC_FEATURE (2)..(2): Xaa = Any amino acid
DXFVFQ

SEQ ID NO: 13 - protein, 9 aa, Artificial Sequence
  Synthetic Peptide
  MISC_FEATURE (3)..(3): Xaa = Any amino acid
IDXFVYQGA

SEQ ID NO: 14 - protein, 7 aa, Artificial Sequence
  Synthetic Peptide
DCFVYQC

SEQ ID NO: 15 - protein, 17 aa, Artificial Sequence
  Synthetic Peptide
  MISC_FEATURE (1)..(6): Xaa = Any amino acid
  MISC_FEATURE (7)..(7): Xaa = Glutamic acid or Aspartic acid
  MISC_FEATURE (8)..(8): Xaa = Any amino acid
  MISC_FEATURE (11)..(11): Xaa = Tyrosine or Phenylalanine
  MISC_FEATURE (14)..(17): Xaa = Any amino acid
XXXXXXXXFVXQCXXXX

SEQ ID NO: 16 - protein, 15 aa, Artificial Sequence
  Synthetic Peptide
  MISC_FEATURE (1)..(5): Xaa = Any amino acid
  MISC_FEATURE (9)..(9): Xaa = Any amino acid
  MISC_FEATURE (12)..(15): Xaa = Any amino acid
XXXXXPEQXFPXXXX

SEQ ID NO: 17 - protein, 15 aa, Artificial Sequence
  Synthetic Peptide
  MISC_FEATURE (1)..(4): Xaa = Any amino acid
  MISC_FEATURE (6)..(6): Xaa = Serine, Threonine, or Alanine
  MISC_FEATURE (9)..(9): Xaa = Tyrosine or Phenylalanine
  MISC_FEATURE (11)..(15): Xaa = Any amino acid
XXXXDXFVXQXXXXX

SEQ ID NO: 18 - skipped

SEQ ID NO: 19 - DNA, 18 bp, Artificial Sequence
  Synthetic Primer
ggctgaaaatcttctctc

SEQ ID NO: 20 - DNA, 94 bp, Artificial Sequence
  Synthetic Primer
  misc_feature (28)..(29): N = Any nucleotide
  misc_feature (30)..(30): S = Guanine or Cytosine
  misc_feature (31)..(32): N = Any nucleotide
  misc_feature (33)..(33): S = Guanine or Cytosine
  misc_feature (34)..(35): N = Any nucleotide
  misc_feature (36)..(36): S = Guanine or Cytosine
  misc_feature (37)..(38): N = Any nucleotide
  misc_feature (39)..(39): S = Guanine or Cytosine
  misc_feature (40)..(41): N = Any nucleotide
  misc_feature (42)..(42): S = Guanine or Cytosine
  misc_feature (52)..(53): N = Any nucleotide
  misc_feature (54)..(54): S = Guanine or Cytosine
  misc_feature (61)..(62): N = Any nucleotide
  misc_feature (63)..(63): S = Guanine or Cytosine
  misc_feature (64)..(65): N = Any nucleotide
  misc_feature (66)..(66): S = Guanine or Cytosine
  misc_feature (67)..(68): N = Any nucleotide
  misc_feature (69)..(69): S = Guanine or Cytosine
  misc_feature (70)..(71): N = Any nucleotide
  misc_feature (72)..(72): S = Guanine or Cytosine
acttccgtagctggccagtctggccagnnsnnsnnsnnsnnsccggaacagnnstttccgnnsnnsnnsnnsggagggcagtctgggcagtctg

SEQ ID NO: 21 - DNA, 18 bp, Artificial Sequence
  Synthetic Primer
ggctgaaaatcttctctc

SEQ ID NO: 22 - protein, 6 aa, Artificial Sequence
  Synthetic Peptide
  MISC_FEATURE (2)..(2): X = Any amino acid
  MISC_FEATURE (5)..(5): X = Phenylalanine or Tyrosine
DXFVXQ

SEQ ID NO: 23 - protein, 9 aa, Artificial Sequence
  Synthetic Peptide
  MISC_FEATURE (2)..(2): X = Any amino acid
  MISC_FEATURE (4)..(4): X = Any amino acid
CXDXFVYQC

SEQ ID NO: 24 - protein, 9 aa, Artificial Sequence
  Synthetic Peptide
PEQLPQFEE

SEQ ID NO: 25 - protein, 8 aa, Artificial Sequence
  Synthetic Peptide
  MISC_FEATURE (5)..(5): Xaa = Alanine or Serine
QPEQXFPE

SEQ ID NO: 26 - protein, 12 aa, Artificial Sequence
  Synthetic Peptide
  MISC_FEATURE (2)..(2): Xaa = Proline, Arginine, or Methionine
  MISC_FEATURE (4)..(4): Xaa = Proline or Alanine
  MISC_FEATURE (5)..(5): Xaa = Glutamine or Phenylalanine
  MISC_FEATURE (9)..(9): Xaa = Alanine or Proline
  MISC_FEATURE (12)..(12): Xaa = Glutamic acid or Aspartic acid
PXEXXPEQXFPX

SEQ ID NO: 27 - protein, 17 aa, Artificial Sequence
  Synthetic Peptide
  MISC_FEATURE (1)..(6): Xaa = Any amino acid
  MISC_FEATURE (7)..(7): Xaa = Glutamic acid or Aspartic acid
  MISC_FEATURE (8)..(8): Xaa = Any amino acid
  MISC_FEATURE (11)..(11): Xaa = Tyrosine or Phenylalanine
  MISC_FEATURE (14)..(17): Xaa = Any amino acid
XXXXXXXXFVXQCXXXX

SEQ ID NO: 28 - protein, 9 aa, Artificial Sequence
  Synthetic Peptide
  MISC_FEATURE (4)..(4): Xaa = Serine or threonine
  MISC_FEATURE (7)..(7): Xaa = Phenylalanine or Tyrosine
CRDXFVXQC

SEQ ID NO: 29 - protein, 8 aa, Artificial Sequence
  Synthetic Peptide
  MISC_FEATURE (6)..(6): Xaa = Any amino acid
PFPEQXFP

SEQ ID NO: 30 - protein, 10 aa, Artificial Sequence
  Synthetic Peptide
  MISC_FEATURE (3)..(3): Xaa = Any amino acid
  MISC_FEATURE (5)..(5): Xaa = Serine or Threonine
  MISC_FEATURE (8)..(8): Xaa = Phenylalanine or Tyrosine
RCXDXFVXQC

SEQ ID NO: 31 - protein, 7 aa, Artificial Sequence
  Synthetic Peptide
  MISC_FEATURE (5)..(5): Xaa = Phenylalanine or Tyrosine
DCFVXQC

SEQ ID NO: 32 - protein, 16 aa, Artificial Sequence
  Synthetic Peptide
  MISC_FEATURE (1)..(6): Xaa = Any amino acid
  MISC_FEATURE (8)..(8): Xaa = Serine, Threonine, or Alanine
  MISC_FEATURE (11)..(11): Xaa = Phenylalanine or Tyrosine
  MISC_FEATURE (13)..(16): Xaa = Any amino acid
XXXXXXDXFVXQXXXX

SEQ ID NO: 33 - protein, 9 aa, Artificial Sequence
  Synthetic Peptide
  MISC_FEATURE (7)..(7): Xaa = Phenylalanine or Tyrosine
CEDSFVXQC

SEQ ID NO: 34 - protein, 7 aa, Artificial Sequence
  Synthetic Peptide
  MISC_FEATURE (1)..(1): Xaa = Leucine, Isoleucine, Methionine, Phenylalanine, or Glutamic acid
  MISC_FEATURE (3)..(3): Xaa = Serine or Threonine
  MISC_FEATURE (6)..(6): Xaa = Phenylalanine or Tyrosine
XDXFVXQ

SEQ ID NO: 35 - protein, 6 aa, Artificial Sequence
  Synthetic Peptide
  MISC_FEATURE (2)..(2): Xaa = Serine or Threonine
  MISC_FEATURE (5)..(5): Xaa = Phenylalanine or Tyrosine
DXFVXQ

SEQ ID NO: 36 - protein, 15 aa, Artificial Sequence
  Synthetic Peptide
GPQPFPEQLFPDPFR

SEQ ID NO: 37 - protein, 12 aa, Artificial Sequence
  Synthetic Peptide
  MISC_FEATURE (2)..(2): Xaa = Arginine, Methionine, or Proline
  MISC_FEATURE (5)..(5): Xaa = Glutamine or Phenylalanine
  MISC_FEATURE (9)..(9): Xaa = Alanine or Proline
PXEPXPEQXFPE

SEQ ID NO: 38 - protein, 15 aa, Artificial Sequence
  Synthetic Peptide
QRCIDTFVFQCSVSA

SEQ ID NO: 39 - protein, 14 aa, Artificial Sequence
  Synthetic Peptide
QNGIDMFVYQGALA

SEQ ID NO: 40 - protein, 20 aa, Artificial Sequence
  Synthetic Peptide
GQSGQSVAGQPEQAFPDRVM

SEQ ID NO: 41 - protein, 20 aa, Artificial Sequence
  Synthetic Peptide
GQSGQGARTQPEQAFPELYG

SEQ ID NO: 42 - protein, 40 aa, Artificial Sequence
  Synthetic Peptide
GQSGQSLLAQPEQGFPEQWRGQSGQSLLAQPEQGFPEQWR

SEQ ID NO: 43 - protein, 20 aa, Artificial Sequence
  Synthetic Peptide
GQSGQRWTNQPEQAFPDRSS

SEQ ID NO: 44 - protein, 20 aa, Artificial Sequence
  Synthetic Peptide
GQSGQGGLGQPEQSFPERVK

SEQ ID NO: 45 - protein, 20 aa, Artificial Sequence
  Synthetic Peptide
GQSGQRRGAQPEQSFPDLGM

SEQ ID NO: 46 - protein, 20 aa, Artificial Sequence
  Synthetic Peptide
GQSGQGSLAQPEQAFPDEKR

SEQ ID NO: 47 - protein, 20 aa, Artificial Sequence
  Synthetic Peptide
GQSGQSVTAFPEQAFPMIRG

SEQ ID NO: 48 - protein, 20 aa, Artificial Sequence
  Synthetic Peptide
GQSGQGWSAFPEQEFPLRQQ

SEQ ID NO: 49 - protein, 20 aa, Artificial Sequence
  Synthetic Peptide
GQSGQVSQAFPEQEFPRIVK

SEQ ID NO: 50 - protein, 20 aa, Artificial Sequence
  Synthetic Peptide
GQSGQKIYSQPEQGFPEKWM

SEQ ID NO: 51 - protein, 30 aa, Artificial Sequence
  Synthetic Peptide
GQSGQPEQAFPDGLSGQSGQPEQAFPDGLS

SEQ ID NO: 52 - protein, 20 aa, Artificial Sequence
  Synthetic Peptide
GQSGQRPGWQPEQAFPEGIR

SEQ ID NO: 53 - protein, 20 aa, Artificial Sequence
  Synthetic Peptide
GQSGQGGSSQPEQAFPEYRV

SEQ ID NO: 54 - protein, 60 aa, Artificial Sequence
  Synthetic Peptide
GQSGQSAWSQPEQSFPELGHGQSGQSAWSQPEQSFPELGHGQSGQSAWSQPEQSFPELGH

SEQ ID NO: 55 - protein, 20 aa, Artificial Sequence
  Synthetic Peptide
GQSGQRAGIQPEQSFPEVGL

SEQ ID NO: 56 - protein, 20 aa, Artificial Sequence
  Synthetic Peptide
GQSGQPQTPFPEQVFPSLGQ

SEQ ID NO: 57 - protein, 20 aa, Artificial Sequence
  Synthetic Peptide
GQSGQPAAPFPEQEFPTGHR

SEQ ID NO: 58 - protein, 20 aa, Artificial Sequence
  Synthetic Peptide
GQSGQVSVRGPEQPFPRLIS

SEQ ID NO: 59 - protein, 19 aa, Artificial Sequence
  Synthetic Peptide
GQSGQVMNSQPEQSFPDAR

SEQ ID NO: 60 - protein, 18 aa, Artificial Sequence
  Synthetic Peptide
GQSGQAGLVSPEQPFPLS

SEQ ID NO: 61 - protein, 20 aa, Artificial Sequence
  Synthetic Peptide
GQSGQRLGAGPEQPFPVRSF

SEQ ID NO: 62 - protein, 20 aa, Artificial Sequence
  Synthetic Peptide
GQSGQVGPVLPEQSFPGWMG

SEQ ID NO: 63 - protein, 20 aa, Artificial Sequence
  Synthetic Peptide
GQSGQGFKLGPEQRFPFMEV

SEQ ID NO: 64 - protein, 15 aa, Artificial Sequence
  Synthetic Peptide
GQSGQPEQGFPSSWN

SEQ ID NO: 65 - protein, 20 aa, Artificial Sequence
  Synthetic Peptide
GQSGQPVQSFPEQLFPRGGS

SEQ ID NO: 66 - protein, 20 aa, Artificial Sequence
  Synthetic Peptide
GQSGQPGIAFPEQQFPNLPG

SEQ ID NO: 67 - protein, 20 aa, Artificial Sequence
  Synthetic Peptide
GQSGQVMIKAPEQAFPGRGL

SEQ ID NO: 68 - protein, 20 aa, Artificial Sequence
  Synthetic Peptide
GQSGQRYRVQPEQGFPEGMA

SEQ ID NO: 69 - protein, 20 aa, Artificial Sequence
  Synthetic Peptide
GQSGQGLLMSPEQGFPFRER

SEQ ID NO: 70 - protein, 20 aa, Artificial Sequence
  Synthetic Peptide
GQSGQVLMEGPEQAFPGAAK

SEQ ID NO: 71 - protein, 20 aa, Artificial Sequence
  Synthetic Peptide
GQSGQKIYSQPEQGFPEKWM

SEQ ID NO: 72 - protein, 20 aa, Artificial Sequence
  Synthetic Peptide
  MISC_FEATURE (1)..(5): Xaa = Any amino acid
XXXXXGQSGQPEQAFPDGLS

SEQ ID NO: 73 - protein, 20 aa, Artificial Sequence
  Synthetic Peptide
GQSGQRPGWQPEQAFPEGIR

SEQ ID NO: 74 - protein, 20 aa, Artificial Sequence
  Synthetic Peptide
GQSGQGGSSQPEQAFPEYRV

SEQ ID NO: 75 - protein, 20 aa, Artificial Sequence
  Synthetic Peptide
GQSGQSAWSQPEQSFPELGH

SEQ ID NO: 76 - protein, 20 aa, Artificial Sequence
  Synthetic Peptide
GQSGQPYRGSPEQPFPVLEG

SEQ ID NO: 77 - protein, 20 aa, Artificial Sequence
  Synthetic Peptide
GQSGQGQRFMPEQAFPMIGM

SEQ ID NO: 78 - protein, 20 aa, Artificial Sequence
  Synthetic Peptide
GQSGQPVVAFPEQLFPKLLK

SEQ ID NO: 79 - protein, 20 aa, Artificial Sequence
  Synthetic Peptide
GQSGQELRVQPEQSFPESLR

SEQ ID NO: 80 - protein, 20 aa, Artificial Sequence
  Synthetic Peptide
GQSGQSTRSNPEQPFPSGVL

SEQ ID NO: 81 - protein, 20 aa, Artificial Sequence
  Synthetic Peptide
GQSGQSGWRLPEQAFPMQMN

SEQ ID NO: 82 - protein, 20 aa, Artificial Sequence
  Synthetic Peptide
GQSGQSLEAFPEQGFPGPRE

SEQ ID NO: 83 - protein, 20 aa, Artificial Sequence
  Synthetic Peptide
GQSGQRVAWTPEQSFPFPEK

SEQ ID NO: 84 - protein, 20 aa, Artificial Sequence
  Synthetic Peptide
GQSGQRARIMPEQAFPGGLI

SEQ ID NO: 85 - protein, 20 aa, Artificial Sequence
  Synthetic Peptide
GQSGQSGTGNPEQPFPQLDR

SEQ ID NO: 86 - protein, 20 aa, Artificial Sequence
  Synthetic Peptide
GQSGQGLMGQPEQSFPEVVS

SEQ ID NO: 87 - protein, 20 aa, Artificial Sequence
  Synthetic Peptide
GQSGQSVAGQPEQAFPDRVM

SEQ ID NO: 88 - protein, 20 aa, Artificial Sequence
  Synthetic Peptide
GQSGQGARTQPEQAFPELYG

SEQ ID NO: 89 - protein, 20 aa, Artificial Sequence
  Synthetic Peptide
GQSGQSLLAQPEQGFPEQWR

SEQ ID NO: 90 - protein, 20 aa, Artificial Sequence
  Synthetic Peptide
GQSGQRWTNQPEQAFPDRSS

SEQ ID NO: 91 - protein, 20 aa, Artificial Sequence
  Synthetic Peptide
GQSGQGGLGQPEQSFPERVK

SEQ ID NO: 92 - protein, 20 aa, Artificial Sequence
  Synthetic Peptide
GQSGQRRGAQPEQSFPDLGM

SEQ ID NO: 93 - protein, 20 aa, Artificial Sequence
  Synthetic Peptide
GQSGQRAGIQPEQSFPEVGL

SEQ ID NO: 94 - protein, 20 aa, Artificial Sequence
  Synthetic Peptide
GQSGQSVTAFPEQAFPMIRG

SEQ ID NO: 95 - protein, 20 aa, Artificial Sequence
  Synthetic Peptide
GQSGQGWSAFPEQEFPLRQQ

SEQ ID NO: 96 - protein, 20 aa, Artificial Sequence
  Synthetic Peptide
GQSGQVSQAFPEQEFPRIVK

SEQ ID NO: 97 - protein, 20 aa, Artificial Sequence
  Synthetic Peptide
  MISC_FEATURE (20)..(20): Xaa = Any amino acid
GQSGQVMNSQPEQSFPDARX

SEQ ID NO: 98 - protein, 20 aa, Artificial Sequence
  Synthetic Peptide
  MISC_FEATURE (19)..(20): Xaa = Any amino acid
GQSGQAGLVSPEQPFPLSXX

SEQ ID NO: 99 - protein, 20 aa, Artificial Sequence
  Synthetic Peptide
GQSGQRLGAGPEQPFPVRSF

SEQ ID NO: 100 - protein, 20 aa, Artificial Sequence
  Synthetic Peptide
GQSGQVGPVLPEQSFPGWMG

SEQ ID NO: 101 - protein, 20 aa, Artificial Sequence
  Synthetic Peptide
GQSGQGFKLGPEQRFPFMEV

SEQ ID NO: 102 - protein, 20 aa, Artificial Sequence
  Synthetic Peptide
  MISC_FEATURE (16)..(20): Xaa = Any amino acid
GQSGQPEQGFPSSWNXXXXX

SEQ ID NO: 103 - protein, 20 aa, Artificial Sequence
  Synthetic Peptide
GQSGQPVQSFPEQLFPRGGS

SEQ ID NO: 104 - protein, 20 aa, Artificial Sequence
  Synthetic Peptide
GQSGQPGIAFPEQQFPNLPG

SEQ ID NO: 105 - protein, 20 aa, Artificial Sequence
  Synthetic Peptide
GQSGQVMIKAPEQAFPGRGL

SEQ ID NO: 106 - protein, 20 aa, Artificial Sequence
  Synthetic Peptide
GQSGQRYRVQPEQGFPEGMA

SEQ ID NO: 107 - protein, 20 aa, Artificial Sequence
  Synthetic Peptide
GQSGQGLLMSPEQGFPFRER

SEQ ID NO: 108 - protein, 20 aa, Artificial Sequence
  Synthetic Peptide
GQSGQVLMEGPEQAFPGAAK

SEQ ID NO: 109 - protein, 20 aa, Artificial Sequence
  Synthetic Peptide
GQSGQGSLAQPEQAFPDEKR

SEQ ID NO: 110 - protein, 20 aa, Artificial Sequence
  Synthetic Peptide
GQSGQPQTPFPEQVFPSLGQ

SEQ ID NO: 111 - protein, 20 aa, Artificial Sequence
  Synthetic Peptide
GQSGQPAAPFPEQEFPTGHR

SEQ ID NO: 112 - protein, 20 aa, Artificial Sequence
  Synthetic Peptide
GQSGQVSVRGPEQPFPRLIS

SEQ ID NO: 113 - protein, 20 aa, Artificial Sequence
  Synthetic Peptide
GQSGQPYRGSPEQPFPVLEG

SEQ ID NO: 114 - protein, 20 aa, Artificial Sequence
  Synthetic Peptide
GQSGQGQRFMPEQAFPMIGM

SEQ ID NO: 115 - protein, 20 aa, Artificial Sequence
  Synthetic Peptide
GQSGQPVVAFPEQLFPKLLK

SEQ ID NO: 116 - protein, 20 aa, Artificial Sequence
  Synthetic Peptide
GQSGQELRVQPEQSFPESLR

SEQ ID NO: 117 - protein, 20 aa, Artificial Sequence
  Synthetic Peptide
GQSGQSTRSNPEQPFPSGVL

SEQ ID NO: 118 - protein, 20 aa, Artificial Sequence
  Synthetic Peptide
GQSGQSGWRLPEQAFPMQMN

SEQ ID NO: 119 - protein, 20 aa, Artificial Sequence
  Synthetic Peptide
GQSGQSLEAFPEQGFPGPRE

SEQ ID NO: 120 - protein, 20 aa, Artificial Sequence
  Synthetic Peptide
GQSGQRVAWTPEQSFPFPEK

SEQ ID NO: 121 - protein, 20 aa, Artificial Sequence
  Synthetic Peptide
GQSGQRARIMPEQAFPGGLI

SEQ ID NO: 122 - protein, 20 aa, Artificial Sequence
  Synthetic Peptide
GQSGQSGTGNPEQPFPQLDR

SEQ ID NO: 123 - protein, 20 aa, Artificial Sequence
  Synthetic Peptide
GQSGQGLMGQPEQSFPEVVS

SEQ ID NO: 124 - protein, 15 aa, Artificial Sequence
  Synthetic Peptide
GHVRDSFVYQTFGGE

SEQ ID NO: 125 - protein, 15 aa, Artificial Sequence
  Synthetic Peptide
VDLRDTFVYQERSLR

SEQ ID NO: 126 - protein, 15 aa, Artificial Sequence
  Synthetic Peptide
SGLRDSFVYQEEGSS

SEQ ID NO: 127 - protein, 15 aa, Artificial Sequence
  Synthetic Peptide
IVMLDSFVYQDFKGR

SEQ ID NO: 128 - protein, 15 aa, Artificial Sequence
  Synthetic Peptide
GPVVDSFVYQTGSYI

SEQ ID NO: 129 - protein, 15 aa, Artificial Sequence
  Synthetic Peptide
SIVLDTFVFQGGQDR

SEQ ID NO: 130 - protein, 15 aa, Artificial Sequence
  Synthetic Peptide
VRPLDSFVYQQLSKE

SEQ ID NO: 131 - protein, 15 aa, Artificial Sequence
  Synthetic Peptide
MHITDSFVFQDGTNV

SEQ ID NO: 132 - protein, 15 aa, Artificial Sequence
  Synthetic Peptide
RRHSDSFVFQTEASA

SEQ ID NO: 133 - protein, 15 aa, Artificial Sequence
  Synthetic Peptide
VIHADSFVFQGGLKE

SEQ ID NO: 134 - protein, 15 aa, Artificial Sequence
  Synthetic Peptide
PCASDTFVFQDRKCG

SEQ ID NO: 135 - protein, 15 aa, Artificial Sequence
  Synthetic Peptide
RLGFDSFVFQRYPKK

SEQ ID NO: 136 - protein, 15 aa, Artificial Sequence
  Synthetic Peptide
YRSRDTFVYQNSSKR

SEQ ID NO: 137 - protein, 15 aa, Artificial Sequence
  Synthetic Peptide
RLVRDSFVYQEKVYE

SEQ ID NO: 138 - protein, 15 aa, Artificial Sequence
  Synthetic Peptide
FRGRDTFVYQTGSQM

SEQ ID NO: 139 - protein, 15 aa, Artificial Sequence
  Synthetic Peptide
TLRLDTFVFQPGDDM

SEQ ID NO: 140 - protein, 11 aa, Artificial Sequence
  Synthetic Peptide
RGDVDSFVFQS

SEQ ID NO: 141 - protein, 15 aa, Artificial Sequence
  Synthetic Peptide
SLALDSFVYQGSRYQ

SEQ ID NO: 142 - protein, 15 aa, Artificial Sequence
  Synthetic Peptide
YGRQDSFVFRVLQLG

SEQ ID NO: 143 - protein, 15 aa, Artificial Sequence
  Synthetic Peptide
SGTSDSFVFQEGENR

SEQ ID NO: 144 - protein, 15 aa, Artificial Sequence
  Synthetic Peptide
FIDADSFVYQRHTQL

SEQ ID NO: 145 - protein, 15 aa, Artificial Sequence
  Synthetic Peptide
LSFMDTFVYQGTPAL

SEQ ID NO: 146 - protein, 15 aa, Artificial Sequence
  Synthetic Peptide
VRVEDTFVYQGRALL

SEQ ID NO: 147 - protein, 15 aa, Artificial Sequence
  Synthetic Peptide
LVGLDSFVYQMGPVK

SEQ ID NO: 148 - protein, 15 aa, Artificial Sequence
  Synthetic Peptide
NTILDSFVYQGSVHP

SEQ ID NO: 149 - protein, 15 aa, Artificial Sequence
  Synthetic Peptide
HGRLDTFVYQPTLGR

SEQ ID NO: 150 - protein, 15 aa, Artificial Sequence
  Synthetic Peptide
LFQLDSFVYQGSLGW

SEQ ID NO: 151 - protein, 15 aa, Artificial Sequence
  Synthetic Peptide
PRWRDSFVYQHAMIE

SEQ ID NO: 152 - protein, 15 aa, Artificial Sequence
  Synthetic Peptide
CHVRDAFVYQGSCRL

SEQ ID NO: 153 - protein, 14 aa, Artificial Sequence
  Synthetic Peptide
RQLRDSFVYQVVGN

SEQ ID NO: 154 - protein, 15 aa, Artificial Sequence
  Synthetic Peptide
YSGFDSFVFQRTGSG

SEQ ID NO: 155 - protein, 15 aa, Artificial Sequence
  Synthetic Peptide
KRCSDSFVFQRSSYC

SEQ ID NO: 156 - protein, 15 aa, Artificial Sequence
  Synthetic Peptide
QCAEDSFVFQACSGG

SEQ ID NO: 157 - protein, 15 aa, Artificial Sequence
  Synthetic Peptide
VQAYDSFVFQHVGYS

SEQ ID NO: 158 - protein, 15 aa, Artificial Sequence
  Synthetic Peptide
VRLVDSFVFQGAAHL

SEQ ID NO: 159 - protein, 15 aa, Artificial Sequence
  Synthetic Peptide
RNGLDTFVYQDAVVH

SEQ ID NO: 160 - protein, 15 aa, Artificial Sequence
  Synthetic Peptide
RYWLDSFVFQAPGCC

SEQ ID NO: 161 - protein, 11 aa, Artificial Sequence
  Synthetic Peptide
GPLRDTFVFQR

SEQ ID NO: 162 - protein, 14 aa, Artificial Sequence
  Synthetic Peptide
WACRDAFVYQDGCK

SEQ ID NO: 163 - protein, 15 aa, Artificial Sequence
  Synthetic Peptide
HTTRDSFVYQLEGER

SEQ ID NO: 164 - protein, 15 aa, Artificial Sequence
  Synthetic Peptide
PRFRDSFVFQEQKLS

SEQ ID NO: 165 - protein, 15 aa, Artificial Sequence
  Synthetic Peptide
LIGDDTFVYQHVGQF

SEQ ID NO: 166 - protein, 15 aa, Artificial Sequence
  Synthetic Peptide
ARAADSFVFQYGRGE

SEQ ID NO: 167 - protein, 15 aa, Artificial Sequence
  Synthetic Peptide
VDLSDSFVYQSLTRK

SEQ ID NO: 168 - protein, 15 aa, Artificial Sequence
  Synthetic Peptide
RCKNDSFVFQACSPH

SEQ ID NO: 169 - protein, 15 aa, Artificial Sequence
  Synthetic Peptide
SYCLDSFVYQSASCS

SEQ ID NO: 170 - protein, 15 aa, Artificial Sequence
  Synthetic Peptide
CDGIDTFVYQCSSNL

SEQ ID NO: 171 - protein, 14 aa, Artificial Sequence
  Synthetic Peptide
ALSKDTFVFQILAH

SEQ ID NO: 172 - protein, 15 aa, Artificial Sequence
  Synthetic Peptide
RIGLDSFVFQNPRVT

SEQ ID NO: 173 - protein, 15 aa, Artificial Sequence
  Synthetic Peptide
NSAIDTFVFQSPTNP

SEQ ID NO: 174 - protein, 15 aa, Artificial Sequence
  Synthetic Peptide
PRVMDSFVYQQVISL

SEQ ID NO: 175 - protein, 15 aa, Artificial Sequence
  Synthetic Peptide
ICESDTFVFQGSCRG

SEQ ID NO: 176 - protein, 15 aa, Artificial Sequence
  Synthetic Peptide
RESEDSFVYQQTRRV

SEQ ID NO: 177 - protein, 14 aa, Artificial Sequence
  Synthetic Peptide
RLVGDAFVFQRSSD

SEQ ID NO: 178 - protein, 15 aa, Artificial Sequence
  Synthetic Peptide
GTGTDSFVYQSTEPG

SEQ ID NO: 179 - protein, 15 aa, Artificial Sequence
  Synthetic Peptide
CEGMDSFVYQCFSQY

SEQ ID NO: 180 - protein, 15 aa, Artificial Sequence
  Synthetic Peptide
SCIMDSFVYQGSQCL

SEQ ID NO: 181 - protein, 15 aa, Artificial Sequence
  Synthetic Peptide
CQTKDSFVYQCHYQE

SEQ ID NO: 182 - protein, 13 aa, Artificial Sequence
  Synthetic Peptide
HRDSFVFQYGGDG

SEQ ID NO: 183 - protein, 15 aa, Artificial Sequence
  Synthetic Peptide
YVELDTFVYQGNSSM

SEQ ID NO: 184 - protein, 14 aa, Artificial Sequence
  Synthetic Peptide
GAAVDSFVFQFPDS

SEQ ID NO: 185 - protein, 15 aa, Artificial Sequence
  Synthetic Peptide
GSRMDSFVYQGFPWN

SEQ ID NO: 186 - protein, 15 aa, Artificial Sequence
  Synthetic Peptide
GLGSDSFVYQSTSYT

SEQ ID NO: 187 - protein, 15 aa, Artificial Sequence
  Synthetic Peptide
RMTDDSFVYQSLGRS

SEQ ID NO: 188 - protein, 15 aa, Artificial Sequence
  Synthetic Peptide
SPLNDTFVYQRFLVP

SEQ ID NO: 189 - protein, 15 aa, Artificial Sequence
  Synthetic Peptide
MKRYDAFVFQDVQPL

SEQ ID NO: 190 - protein, 15 aa, Artificial Sequence
  Synthetic Peptide
GLCNDTFVYQDKCGS

SEQ ID NO: 191 - protein, 15 aa, Artificial Sequence
  Synthetic Peptide
HGCEDTFVYQCGNAR

SEQ ID NO: 192 - protein, 15 aa, Artificial Sequence
  Synthetic Peptide
VRCVDSFVFQCTKRA

SEQ ID NO: 193 - protein, 15 aa, Artificial Sequence
  Synthetic Peptide
CHTKDAFVFQCDSNL

SEQ ID NO: 194 - protein, 15 aa, Artificial Sequence
  Synthetic Peptide
CFRADAFVYQGCDVM

SEQ ID NO: 195 - protein, 15 aa, Artificial Sequence
  Synthetic Peptide
IGMRDAFVYQIPNLH

SEQ ID NO: 196 - protein, 15 aa, Artificial Sequence
  Synthetic Peptide
SLVLDSFVYQTGDRR

SEQ ID NO: 197 - protein, 15 aa, Artificial Sequence
  Synthetic Peptide
QGIIDSFVYQRSDPG

SEQ ID NO: 198 - protein, 15 aa, Artificial Sequence
  Synthetic Peptide
TQGSDSFVYQTMERG

SEQ ID NO: 199 - protein, 12 aa, Artificial Sequence
  Synthetic Peptide
ADTFVFQEMHVK

SEQ ID NO: 200 - protein, 15 aa, Artificial Sequence
  Synthetic Peptide
GLCVDTFVYQGQGCR

SEQ ID NO: 201 - protein, 15 aa, Artificial Sequence
  Synthetic Peptide
VECVDSFVYQCKRGS

SEQ ID NO: 202 - protein, 15 aa, Artificial Sequence
  Synthetic Peptide
SCGGDAFVYQCFVSS

SEQ ID NO: 203 - protein, 15 aa, Artificial Sequence
  Synthetic Peptide
TCMLDAFVFQTGLCG

SEQ ID NO: 204 - protein, 15 aa, Artificial Sequence
  Synthetic Peptide
ASYRDSFVFQDHHTG

SEQ ID NO: 205 - protein, 15 aa, Artificial Sequence
  Synthetic Peptide
VERLDTFVYQRVDTR

SEQ ID NO: 206 - protein, 15 aa, Artificial Sequence
  Synthetic Peptide
LRQLDSFVYQGFEIV

SEQ ID NO: 207 - protein, 15 aa, Artificial Sequence
  Synthetic Peptide
LSAIDTFVFQSSPRE

SEQ ID NO: 208 - protein, 15 aa, Artificial Sequence
  Synthetic Peptide
LRWHDTFVYQGSLSP

SEQ ID NO: 209 - protein, 17 aa, Artificial Sequence
  Synthetic Peptide
VDSGCVDSFVYQCRSLG

SEQ ID NO: 210 - protein, 17 aa, Artificial Sequence
  Synthetic Peptide
GFKKCSDSFVYQCMGGK

SEQ ID NO: 211 - protein, 17 aa, Artificial Sequence
  Synthetic Peptide
LNGACGDSFVFQCTAGL

SEQ ID NO: 212 - protein, 17 aa, Artificial Sequence
  Synthetic Peptide
QLVHCYDTFVFQCDAAR

SEQ ID NO: 213 - protein, 15 aa, Artificial Sequence
  Synthetic Peptide
VSPLERDCFVYQCVS

SEQ ID NO: 214 - protein, 17 aa, Artificial Sequence
  Synthetic Peptide
SPLMSGDCFVYQCATHS

SEQ ID NO: 215 - protein, 17 aa, Artificial Sequence
  Synthetic Peptide
AITCSHDAFVYQCGVPM

SEQ ID NO: 216 - protein, 17 aa, Artificial Sequence
  Synthetic Peptide
SACGGVDNFVYQCSLAN

SEQ ID NO: 217 - protein, 17 aa, Artificial Sequence
  Synthetic Peptide
VPGTCQDGFVYQCLWGA

SEQ ID NO: 218 - protein, 17 aa, Artificial Sequence
  Synthetic Peptide
GRACRSDAFVFQCGLSA

SEQ ID NO: 219 - protein, 17 aa, Artificial Sequence
  Synthetic Peptide
LPLTYFELFVFQCMCHM

SEQ ID NO: 220 - protein, 17 aa, Artificial Sequence
  Synthetic Peptide
RTGVCSDSFVYQCDPVA

SEQ ID NO: 221 - protein, 16 aa, Artificial Sequence
  Synthetic Peptide
GSAGCIDSFVFQCGLR

SEQ ID NO: 222 - protein, 17 aa, Artificial Sequence
  Synthetic Peptide
RGDRCSDTFVFQCWTPD

SEQ ID NO: 223 - protein, 17 aa, Artificial Sequence
  Synthetic Peptide
GSRYGRDCFVFQCGVDL

SEQ ID NO: 224 - protein, 17 aa, Artificial Sequence
  Synthetic Peptide
LFGLESDCFVYQCSNTP

SEQ ID NO: 225 - protein, 17 aa, Artificial Sequence
  Synthetic Peptide
RLNSCRDLFVYQCWQAG

SEQ ID NO: 226 - protein, 17 aa, Artificial Sequence
  Synthetic Peptide
LGTCGADPFVFQCQNIR

SEQ ID NO: 227 - protein, 17 aa, Artificial Sequence
  Synthetic Peptide
GVCMARDAFVYQCLRGG

SEQ ID NO: 228 - protein, 17 aa, Artificial Sequence
  Synthetic Peptide
CSGLLMDRFVYQCDVVN

SEQ ID NO: 229 - protein, 17 aa, Artificial Sequence
  Synthetic Peptide
CMSGVIDPFVFQCEGMG

SEQ ID NO: 230 - protein, 13 aa, Artificial Sequence
  Synthetic Peptide
CSETFVFQCPMGA

SEQ ID NO: 231 - protein, 22 aa, Artificial Sequence
  Synthetic Peptide
GQSGQSSVLCRDTFVFQCTPVV

SEQ ID NO: 232 - protein, 22 aa, Artificial Sequence
  Synthetic Peptide
GQSGQWRHGCADSFVFQCDAWQ

SEQ ID NO: 233 - protein, 22 aa, Artificial Sequence
  Synthetic Peptide
GQSGQLRGGCADSFVYQCESSA

SEQ ID NO: 234 - protein, 44 aa, Artificial Sequence
  Synthetic Peptide
GQSGQVCEPFTDSFVYQCPSANGQSGQVCEPFTDSFVYQCPSAN

SEQ ID NO: 235 - protein, 22 aa, Artificial Sequence
  Synthetic Peptide
GQSGQVSPLERDCFVYQCVSSS

SEQ ID NO: 236 - protein, 22 aa, Artificial Sequence
  Synthetic Peptide
GQSGQVLPTSGDCFVYQCDLTM

SEQ ID NO: 237 - protein, 36 aa, Artificial Sequence
  Synthetice Peptide
GQSGQVCQDEFVFQCSSAGQSGQVCQDEFVFQCSSA

SEQ ID NO: 238 - protein, 19 aa, Artificial Sequence
  Synthetic Peptide
GQSGQGCLDEFVFQCAGGS

SEQ ID NO: 239 - protein, 22 aa, Artificial Sequence
  Synthetic Peptide
GQSGQGSGKCIDPFVYQCLRMS

SEQ ID NO: 240 - protein, 22 aa, Artificial Sequence
  Synthetic Peptide
GQSGQLWCTRSDAFVYQCSRMQ

SEQ ID NO: 241 - protein, 19 aa, Artificial Sequence
  Synthetic Peptide
GQSGQGMEAFPEQGFRSPA

SEQ ID NO: 242 - protein, 22 aa, Artificial Sequence
  Synthetic Peptide
GQSGQRYRSCKDSFVFQCGFMP

SEQ ID NO: 243 - protein, 19 aa, Artificial Sequence
  Synthetic Peptide
GQSGQGCRDSFVFQCESGS

SEQ ID NO: 244 - protein, 22 aa, Artificial Sequence
  Synthetic Peptide
GQSGQTVHGCRDSFVYQCESRG

SEQ ID NO: 245 - protein, 22 aa, Artificial Sequence
  Synthetic Peptide
GQSGQVTDRCYDTFVFQCARGS

SEQ ID NO: 246 - protein, 22 aa, Artificial Sequence
  Synthetic Peptide
GQSGQPLSRCIDSFVYQCVSSS

SEQ ID NO: 247 - protein, 22 aa, Artificial Sequence
  Synthetic Peptide
GQSGQAGTRCLDSFVFQCVAVG

SEQ ID NO: 248 - protein, 21 aa, Artificial Sequence
  Synthetic Peptide
GQSGQPELQCFDSFVFQCNGG

SEQ ID NO: 249 - protein, 22 aa, Artificial Sequence
  Synthetic Peptide
GQSGQLVSSEADCFVYQCLSTR

SEQ ID NO: 250 - protein, 22 aa, Artificial Sequence
  Synthetic Peptide
GQSGQSSGSETDCFVYQCGVVL

SEQ ID NO: 251 - protein, 22 aa, Artificial Sequence
  Synthetic Peptide
GQSGQVLPTSGDCFVYQCDLTM

SEQ ID NO: 252 - protein, 22 aa, Artificial Sequence
  Synthetic Peptide
GQSGQGLAQRVDCFVYQCQRGE

SEQ ID NO: 253 - protein, 21 aa, Artificial Sequence
  Synthetic Peptide
GQSGQERRRCLDAFVFQCVEK

SEQ ID NO: 254 - protein, 22 aa, Artificial Sequence
  Synthetic Peptide
GQSGQALQACVDAFVYQCNSGS

SEQ ID NO: 255 - protein, 22 aa, Artificial Sequence
  Synthetic Peptide
GQSGQSLHACYDAFVYQCNSGD

SEQ ID NO: 256 - protein, 19 aa, Artificial Sequence
  Synthetic Peptide
GQSGQGCLDEFVFQCAGGS

SEQ ID NO: 257 - protein, 22 aa, Artificial Sequence
  Synthetic Peptide
GQSGQYGTACVDGFVYQCGVRG

SEQ ID NO: 258 - protein, 22 aa, Artificial Sequence
  Synthetic Peptide
GQSGQNRDRCRDSFVYQCVFPT

SEQ ID NO: 259 - protein, 22 aa, Artificial Sequence
  Synthetic Peptide
GQSGQGTACHADSFVYQCSSQK

SEQ ID NO: 260 - protein, 22 aa, Artificial Sequence
  Synthetic Peptide
GQSGQRSSFCSDSFVFQCELPG

SEQ ID NO: 261 - protein, 22 aa, Artificial Sequence
  Synthetic Peptide
GQSGQRRKCSEDSFVYQCVRST

SEQ ID NO: 262 - protein, 22 aa, Artificial Sequence
  Synthetic Peptide
GQSGQSREGRGDCFVYQCHTSI

SEQ ID NO: 263 - protein, 44 aa, Artificial Sequence
  Synthetic Peptide
GQSGQSSGSETDCFVYQCGVVLGQSGQSSGSETDCFVYQCGVVL

SEQ ID NO: 264 - protein, 22 aa, Artificial Sequence
  Synthetic Peptide
GQSGQRSAACSDEFVYQCRGNT

SEQ ID NO: 265 - protein, 22 aa, Artificial Sequence
  Synthetic Peptide
GQSGQMVRSCYDQFVYQCSQTS

SEQ ID NO: 266 - protein, 22 aa, Artificial Sequence
  Synthetic Peptide
GQSGQGSGRCEDAFVYQCQSFG

SEQ ID NO: 267 - protein, 22 aa, Artificial Sequence
  Synthetic Peptide
GQSGQWAAQCVDGFVYQCGAHL

SEQ ID NO: 268 - protein, 66 aa, Artificial Sequence
  Synthetic Peptide
GQSGQMDVRCRDSFVYQCHVGTGQSGQMDVRCRDSFVYQCHVGTGQSGQMDVRCRDSFVYQCHVGT

SEQ ID NO: 269 - protein, 22 aa, Artificial Sequence
  Synthetic Peptide
GQSGQGVTHCKDSFVYQCISGS

SEQ ID NO: 270 - protein, 22 aa, Artificial Sequence
  Synthetic Peptide
GQSGQSKNDCRDSFVFQCGSRS

SEQ ID NO: 271 - protein, 22 aa, Artificial Sequence
  Synthetic Peptide
GQSGQSPERCSDSFVYQCTSQR

SEQ ID NO: 272 - protein, 44 aa, Artificial Sequence
  Synthetic Peptide
GQSGQDHSRCQDTFVFQCGSREGQSGQDHSRCQDTFVFQCGSRE

SEQ ID NO: 273 - protein, 22 aa, Artificial Sequence
  Synthetic Peptide
GQSGQLAGRCQDSFVFQCVEPT

SEQ ID NO: 274 - protein, 22 aa, Artificial Sequence
  Synthetic Peptide
GQSGQVCEPFTDSFVYQCPSAN

SEQ ID NO: 275 - protein, 22 aa, Artificial Sequence
  Synthetic Peptide
GQSGQRGCNHMDTFVYQCPSGR

SEQ ID NO: 276 - protein, 44 aa, Artificial Sequence
  Synthetic Peptide
GQSGQSGRSGTDCFVYQCNGVDGQSGQSGRSGTDCFVYQCNGVD

SEQ ID NO: 277 - protein, 22 aa, Artificial Sequence
  Synthetic Peptide
GQSGQSSRSSTDCFVFQCVEQG

SEQ ID NO: 278 - protein, 22 aa, Artificial Sequence
  Synthetic Peptide
GQSGQRTGRDRDCFVFQCHLSF

SEQ ID NO: 279 - protein, 18 aa, Artificial Sequence
  Synthetic Peptide
GQSGQCLDAFVYQCTANL

SEQ ID NO: 280 - protein, 22 aa, Artificial Sequence
  Synthetic Peptide
GQSGQGKSCHDDLFVYQCGSRM

SEQ ID NO: 281 - protein, 22 aa, Artificial Sequence
  Synthetic Peptide
GQSGQPKRCHSDAFVFQCLPSG

SEQ ID NO: 282 - protein, 19 aa, Artificial Sequence
  Synthetic Peptide
GQSGQCQGLGDERFVFQCV

SEQ ID NO: 283 - protein, 22 aa, Artificial Sequence
  Synthetic Peptide
GQSGQIHVSCRDDFVYQCAYRQ

SEQ ID NO: 284 - protein, 22 aa, Artificial Sequence
  Synthetic Peptide
GQSGQPDQTCAEPFVYQCARGG

SEQ ID NO: 285 - protein, 17 aa, Artificial Sequence
  Synthetic Peptide
GVGGEAHPEQTFKYDEN

SEQ ID NO: 286 - protein, 15 aa, Artificial Sequence
  Synthetic Peptide
VTNFMPEQTWLGFRP

SEQ ID NO: 287 - protein, 11 aa, Artificial Sequence
  Synthetic Peptide
PEQTFSGSGWH

SEQ ID NO: 288 - protein, 15 aa, Artificial Sequence
  Synthetic Peptide
PGNRDAWIAHPEQRF

SEQ ID NO: 289 - protein, 15 aa, Artificial Sequence
  Synthetic Peptide
TASGGPEQPFGSGQG

SEQ ID NO: 290 - protein, 15 aa, Artificial Sequence
  Synthetic Peptide
AQAPEQARPIGSGGS

SEQ ID NO: 291 - protein, 13 aa, Artificial Sequence
  Synthetic Peptide
PEQLLPQTVVGWL

SEQ ID NO: 292 - protein, 15 aa, Artificial Sequence
  Synthetic Peptide
TMQAAPEQVRPTDRA

SEQ ID NO: 293 - protein, 15 aa, Artificial Sequence
  Synthetic Peptide
AFGPPEQVGPTYGNW

SEQ ID NO: 294 - protein, 15 aa, Artificial Sequence
  Synthetic Peptide
MAAVERPEQLLIEPR

SEQ ID NO: 295 - protein, 12 aa, Artificial Sequence
  Synthetic Peptide
PEQLLPQTVVGW

SEQ ID NO: 296 - protein, 15 aa, Artificial Sequence
  Synthetic Peptide
RVVNMNSRPEQVVEG

SEQ ID NO: 297 - protein, 14 aa, Artificial Sequence
  Synthetic Peptide
RAPEQIMEFGRPWE

SEQ ID NO: 298 - protein, 15 aa, Artificial Sequence
  Synthetic Peptide
GAWHTVGAPEQVQTH

SEQ ID NO: 299 - protein, 15 aa, Artificial Sequence
  Synthetic Peptide
TLVNVRPEQPVYFGG

SEQ ID NO: 300 - protein, 15 aa, Artificial Sequence
  Synthetic Peptide
RVYLGGPEQPLPVVR

SEQ ID NO: 301 - protein, 15 aa, Artificial Sequence
  Synthetic Peptide
RVKLLGPEQPLHWGG

SEQ ID NO: 302 - protein, 15 aa, Artificial Sequence
  Synthetic Peptide
ALVMALPEQPVPRAG

SEQ ID NO: 303 - protein, 15 aa, Artificial Sequence
  Synthetic Peptide
VAWTMGPEQPLVRAL

SEQ ID NO: 304 - protein, 15 aa, Artificial Sequence
  Synthetic Peptide
GQGQAFPEQGSVPIN

SEQ ID NO: 305 - protein, 15 aa, Artificial Sequence
  Synthetic Peptide
QPLTVFPEQEVSNRT

SEQ ID NO: 306 - protein, 15 aa, Artificial Sequence
  Synthetic Peptide
RQGQAFPEQVVQVSH

SEQ ID NO: 307 - protein, 15 aa, Artificial Sequence
  Synthetic Peptide
QPKMSFPEQDQSVIR

SEQ ID NO: 308 - protein, 15 aa, Artificial Sequence
  Synthetic Peptide
QDEIAFPEQGMRWGG

SEQ ID NO: 309 - protein, 15 aa, Artificial Sequence
  Synthetic Peptide
VWDRGVPEQMFPRKG

SEQ ID NO: 310 - protein, 14 aa, Artificial Sequence
  Synthetic Peptide
SVGQWLPEQMFPFA

SEQ ID NO: 311 - protein, 15 aa, Artificial Sequence
  Synthetic Peptide
LGGGERPEQQFPVVW

SEQ ID NO: 312 - protein, 14 aa, Artificial Sequence
  Synthetic Peptide
GGISLGPEQAWPVA

SEQ ID NO: 313 - protein, 14 aa, Artificial Sequence
  Synthetic Peptide
RFAWLGPEQAFPIG

SEQ ID NO: 314 - protein, 15 aa, Artificial Sequence
  Synthetic Peptide
VMEPFPEQGFPGSRA

SEQ ID NO: 315 - protein, 15 aa, Artificial Sequence
  Synthetic Peptide
GPQPFPEQLFPDPFR

SEQ ID NO: 316 - protein, 15 aa, Artificial Sequence
  Synthetic Peptide
AEQPFPEQGFPSGTG

SEQ ID NO: 317 - protein, 14 aa, Artificial Sequence
  Synthetic Peptide
SPEPFPEQGFPGIM

SEQ ID NO: 318 - protein, 15 aa, Artificial Sequence
  Synthetic Peptide
FRMPFPEQRFPRSGG

SEQ ID NO: 319 - protein, 15 aa, Artificial Sequence
  Synthetic Peptide
PNVAFPEQAFPRGAI

SEQ ID NO: 320 - protein, 15 aa, Artificial Sequence
  Synthetic Peptide
SVEAFPEQRFPRAER

SEQ ID NO: 321 - protein, 15 aa, Artificial Sequence
  Synthetic Peptide
PPWAFPEQVFPAVGL

SEQ ID NO: 322 - protein, 15 aa, Artificial Sequence
  Synthetic Peptide
PTDAFPEQSFPRHLV

SEQ ID NO: 323 - protein, 15 aa, Artificial Sequence
  Synthetic Peptide
TLFMQPEQSFPERRG

SEQ ID NO: 324 - protein, 12 aa, Artificial Sequence
  Synthetic Peptide
GQPEQAFPEGMA

SEQ ID NO: 325 - protein, 15 aa, Artificial Sequence
  Synthetic Peptide
RRSEQPEQAFPDGLR

SEQ ID NO: 326 - protein, 15 aa, Artificial Sequence
  Synthetic Peptide
LARVQPEQSFPDFML

SEQ ID NO: 327 - protein, 15 aa, Artificial Sequence
  Synthetic Peptide
RGRAQPEQAFPESVG

SEQ ID NO: 328 - protein, 20 aa, Artificial Sequence
  Synthetic Peptide
GQSGQPEQFMALCDCTRAEW

SEQ ID NO: 329 - protein, 19 aa, Artificial Sequence
  Synthetic Peptide
GQSGQPEQICAIGRVGSCT

SEQ ID NO: 330 - protein, 22 aa, Artificial Sequence
  Synthetic Peptide
GQSGQPEQVCSRMRMTWCHSFG

SEQ ID NO: 331 - protein, 20 aa, Artificial Sequence
  Synthetic Peptide
GQSGQPEQICAIGRVGSCTN

SEQ ID NO: 332 - protein, 22 aa, Artificial Sequence
  Synthetic Peptide
GQSGQPEQLCSSTDDAGCAYRR

SEQ ID NO: 333 - protein, 22 aa, Artificial Sequence
  Synthetic Peptide
GQSGQLPEQCLAHLSGQCGSKG

SEQ ID NO: 334 - protein, 21 aa, Artificial Sequence
  Synthetic Peptide
GQSGQPEQICAIGRVGSCTNG

SEQ ID NO: 335 - protein, 22 aa, Artificial Sequence
  Synthetic Peptide
GQSGQPEQKCIIALNDACRYIE

SEQ ID NO: 336 - protein, 22 aa, Artificial Sequence
  Synthetic Peptide
GQSGQPEQPCAAGMQDSCWLRS

SEQ ID NO: 337 - protein, 20 aa, Artificial Sequence
  Synthetic Peptide
GQSGQPEQICAIGRVGSCTN

SEQ ID NO: 338 - protein, 22 aa, Artificial Sequence
  Synthetic Peptide
GQSGQPEQPCAGGQRRQCLWDW

SEQ ID NO: 339 - protein, 20 aa, Artificial Sequence
  Synthetic Peptide
QSGQPEQMREWHDSDVCSMG

SEQ ID NO: 340 - protein, 20 aa, Artificial Sequence
  Synthetic Peptide
GQSGQPEQLCAVRQSEWCGV

SEQ ID NO: 341 - protein, 22 aa, Artificial Sequence
  Synthetic Peptide
GQSGQTGGGCRQLPEQVCSLYY

SEQ ID NO: 342 - protein, 20 aa, Artificial Sequence
  Synthetic Peptide
GQSGQRVVNMNSRPEQVVEG

SEQ ID NO: 343 - protein, 20 aa, Artificial Sequence
  Synthetic Peptide
GQSGQGAWHTVGAPEQVQTH

SEQ ID NO: 344 - protein, 60 aa, Artificial Sequence
  Synthetic Peptide
GQSGQAFGPPEQVGPTYGNWGQSGQAFGPPEQVGPTYGNWGQSGQAFGPPEQVGPTYGNW

SEQ ID NO: 345 - protein, 20 aa, Artificial Sequence
  Synthetic Peptide
GQSGQRAPEQIMEPGRPWER

SEQ ID NO: 346 - protein, 21 aa, Artificial Sequence
  Synthetic Peptide
GQSGQESVPEQLCPWGVCQGR

SEQ ID NO: 347 - protein, 22 aa, Artificial Sequence
  Synthetic Peptide
GQSGQTGGGCRQLPEQVCSLYY

SEQ ID NO: 348 - protein, 21 aa, Artificial Sequence
  Synthetic Peptide
GQSGQESVPEQLCPWGVCQGR

SEQ ID NO: 349 - protein, 21 aa, Artificial Sequence
  Synthetic Peptide
GQSGQGAEAPEQRMHGCCAVS

SEQ ID NO: 350 - protein, 21 aa, Artificial Sequence
  Synthetic Peptide
GQSGQVPEQTRSRGELDCCWG

SEQ ID NO: 351 - protein, 44 aa, Artificial Sequence
  Synthetic Peptide
GQSGQLRGLCPEQSFTSCRVGRGQSGQLRGLCPEQSFTSCRVGR

SEQ ID NO: 352 - protein, 44 aa, Artificial Sequence
  Synthetic Peptide
GQSGQERLMCPEQAMMYCRWNNGQSGQERLMCPEQAMMYCRWNN

SEQ ID NO: 353 - protein, 22 aa, Artificial Sequence
  Synthetic Peptide
GQSGQAVGVCPEQAFDRCAHVR

SEQ ID NO: 354 - protein, 22 aa, Artificial Sequence
  Synthetic Peptide
GQSGQHLQLCPEQDWVGCGGGR

SEQ ID NO: 355 - protein, 22 aa, Artificial Sequence
  Synthetic Peptide
GQSGQYDPSCPEQMFARCLMPG

SEQ ID NO: 356 - protein, 21 aa, Artificial Sequence
  Synthetic Peptide
GQSGQCPEQVLAWQQPVCKKS

SEQ ID NO: 357 - protein, 22 aa, Artificial Sequence
  Synthetic Peptide
GQSGQSPEQCRAWNRSPCEFMP

SEQ ID NO: 358 - protein, 22 aa, Artificial Sequence
  Synthetic Peptide
GQSGQTPEQCWPRWYGVCSVFP

SEQ ID NO: 359 - protein, 20 aa, Artificial Sequence
  Synthetic Peptide
GQSGQVALWRMTWRGPEQAW

SEQ ID NO: 360 - protein, 21 aa, Artificial Sequence
  Synthetic Peptide
GQSGQNDVPEQRWCNARCSRT

SEQ ID NO: 361 - protein, 20 aa, Artificial Sequence
  Synthetic Peptide
GQSGQDSMPEQLWGQNMHTM

SEQ ID NO: 362 - protein, 20 aa, Artificial Sequence
  Synthetic Peptide
GQSGQEGGLKSTSPEQVWGL

SEQ ID NO: 363 - protein, 20 aa, Artificial Sequence
  Synthetic Peptide
GQSGQRPPEQAWPEEVGMHS

SEQ ID NO: 364 - protein, 20 aa, Artificial Sequence
  Synthetic Peptide
GQSGQEDPEQVWGAVPNLRV

SEQ ID NO: 365 - protein, 20 aa, Artificial Sequence
  Synthetic Peptide
GQSGQGHITMPEQEWSWGNL

SEQ ID NO: 366 - protein, 20 aa, Artificial Sequence
  Synthetic Peptide
GQSGQRVGMGLHWPEQGFPE

SEQ ID NO: 367 - protein, 21 aa, Artificial Sequence
  Synthetic Peptide
GQSGQVLPEQHWQLCRGCVRD

SEQ ID NO: 368 - protein, 22 aa, Artificial Sequence
  Synthetic Peptide
GQSGQGDTQCGMNFMTQCFPEQ

SEQ ID NO: 369 - protein, 21 aa, Artificial Sequence
  Synthetic Peptide
GQSGQTDDPFPDQVNETCLIM

SEQ ID NO: 370 - protein, 20 aa, Artificial Sequence
  Synthetic Peptide
GQSGQVYNDMAPDQACGVGA

SEQ ID NO: 371 - protein, 21 aa, Artificial Sequence
  Synthetic Peptide
GQSGQEHLGADPEQRVACIGS

SEQ ID NO: 372 - protein, 20 aa, Artificial Sequence
  Synthetic Peptide
GQSGQANLMKPEQEMGYLKV

SEQ ID NO: 373 - protein, 22 aa, Artificial Sequence
  Synthetic Peptide
GQSGQRMRGCEGGPEQGCLMAH

SEQ ID NO: 374 - protein, 20 aa, Artificial Sequence
  Synthetic Peptide
GQSGQVYDSECEDSYVFQCW

SEQ ID NO: 375 - protein, 19 aa, Artificial Sequence
  Synthetic Peptide
GQSGQAGRHCANTFVYCSA

SEQ ID NO: 376 - protein, 22 aa, Artificial Sequence
  Synthetic Peptide
GQSGQTWRGCEESFVTQCPDAM

SEQ ID NO: 377 - protein, 44 aa, Artificial Sequence
  Synthetic Peptide
GQSGQESNTCDLFVWQACDGKQGQSGQESNTCDLFVWQACDGKQ

SEQ ID NO: 378 - protein, 132 aa, Artificial Sequence
  Synthetic Peptide
GQSGQAEVACEDNFVYQCSDDWGQSGQAEVACEDNFVYQCSDDWGQSGQAEVACEDNFVYQCSDDWGQSGQAEVACEDNFVYQCSDDWGQSGQAEVACEDNFVYQCSDDWGQSGQAEVACEDNFVYQCSDDW

SEQ ID NO: 379 - protein, 22 aa, Artificial Sequence
  Synthetic Peptide
GQSGQSSASCDMFVYQGCAEFN

SEQ ID NO: 380 - protein, 22 aa, Artificial Sequence
  Synthetic Peptide
GQSGQRQGACVDDYVYQCGHFE

SEQ ID NO: 381 - protein, 44 aa, Artificial Sequence
  Synthetic Peptide
GQSGQGHTACMTDFVHQCFPGTGQSGQGHTACMTDFVHQCFPGT

SEQ ID NO: 382 - protein, 21 aa, Artificial Sequence
  Synthetic Peptide
GQSGQPCVDAFVYQQSGCNIA

SEQ ID NO: 383 - protein, 23 aa, Artificial Sequence
  Synthetic Peptide
GQSGQGRAACVDDFVYQCVRQHE

SEQ ID NO: 384 - protein, 22 aa, Artificial Sequence
  Synthetic Peptide
GQSGQNTVVCLDGFVFQCNEWA

SEQ ID NO: 385 - protein, 21 aa, Artificial Sequence
  Synthetic Peptide
GQSGQSQWGCMDGFVWQCGGK

SEQ ID NO: 386 - protein, 22 aa, Artificial Sequence
  Synthetic Peptide
GQSGQGDTFCRDSFVYQCPRFY

SEQ ID NO: 387 - protein, 40 aa, Artificial Sequence
  Synthetic Peptide
GQSGQVEDVCFDGFVFQCTGGQSGQVEDVCFDGFVFQCTG

SEQ ID NO: 388 - protein, 22 aa, Artificial Sequence
  Synthetic Peptide
GQSGQALGDCDSFVFQVCEGRD

SEQ ID NO: 389 - protein, 22 aa, Artificial Sequence
  Synthetic Peptide
GQSGQGLRMCTDSFVNQCELWP

SEQ ID NO: 390 - protein, 44 aa, Artificial Sequence
  Synthetic Peptide
GQSGQRDGHCADSFVNQCVRPLGQSGQRDGHCADSFVNQCVRPL

SEQ ID NO: 391 - protein, 22 aa, Artificial Sequence
  Synthetic Peptide
GQSGQSYPSCLESFVFQCTPDW

SEQ ID NO: 392 - protein, 22 aa, Artificial Sequence
  Synthetic Peptide
GQSGQTGRLCRESFVYQCVNKW

SEQ ID NO: 393 - protein, 17 aa, Artificial Sequence
  Synthetic Peptide
GDTFCRDSFVYQCPRFY

SEQ ID NO: 394 - protein, 17 aa, Artificial Sequence
  Synthetic Peptide
ALGDCDSFVFQVCEGRD

SEQ ID NO: 395 - protein, 17 aa, Artificial Sequence
  Synthetic Peptide
TGRLCRESFVYQCVNKW

SEQ ID NO: 396 - protein, 17 aa, Artificial Sequence
  Synthetic Peptide
TGRLCRESFVYQCVNKW

SEQ ID NO: 397 - protein, 17 aa, Artificial Sequence
  Synthetic Peptide
NTVVCLDGFVFQCNEWA

SEQ ID NO: 398 - protein, 16 aa, Artificial Sequence
  Synthetic Peptide
SQWGCMDGFVWQCGGK

SEQ ID NO: 399 - protein, 15 aa, Artificial Sequence
  Synthetic Peptide
VEDVCFDGFVFQCTG

SEQ ID NO: 400 - protein, 17 aa, Artificial Sequence
  Synthetic Peptide
ESNTCDLFVWQACDGKQ

SEQ ID NO: 401 - protein, 17 aa, Artificial Sequence
  Synthetic Peptide
AEVACEDNFVYQCSDDW

SEQ ID NO: 402 - protein, 17 aa, Artificial Sequence
  Synthetic Peptide
SSASCDMFVYQGCAEFN

SEQ ID NO: 403 - protein, 17 aa, Artificial Sequence
  Synthetic Peptide
RQGACVDDYVYQCGHFE

SEQ ID NO: 404 - protein, 18 aa, Artificial Sequence
  Synthetic Peptide
GRAACVDDFVYQCVRQHE

SEQ ID NO: 405 - protein, 16 aa, Artificial Sequence
  Synthetic Peptide
PCVDAFVYQQSGCNIA

SEQ ID NO: 406 - protein, 15 aa, Artificial Sequence
  Synthetic Peptide
AGRHCANTFVYQCSA

SEQ ID NO: 407 - protein, 17 aa, Artificial Sequence
  Synthetic Peptide
RDGHCADSFVNQCVRPL

SEQ ID NO: 408 - protein, 17 aa, Artificial Sequence
  Synthetic Peptide
GLRMCTDSFVNQCELWP

SEQ ID NO: 409 - protein, 17 aa, Artificial Sequence
  Synthetic Peptide
RDGHCADSFVNQCVRPL

SEQ ID NO: 410 - protein, 17 aa, Artificial Sequence
  Synthetic Peptide
TWRGCEESFVTQCPDAM

SEQ ID NO: 411 - protein, 17 aa, Artificial Sequence
  Synthetic Peptide
GHTACMTDFVHQCFPGT

SEQ ID NO: 412 - protein, 17 aa, Artificial Sequence
  Synthetic Peptide
MDVRCRDSFVYQCHVGT

SEQ ID NO: 413 - protein, 17 aa, Artificial Sequence
  Synthetic Peptide
GQSGCRDSFVFQCESGS

SEQ ID NO: 414 - protein, 17 aa, Artificial Sequence
  Synthetic Peptide
SKNDCRDSFVFQCGSRS

SEQ ID NO: 415 - protein, 17 aa, Artificial Sequence
  Synthetic Peptide
TVHGCRDSFVYQCESRG

SEQ ID NO: 416 - protein, 17 aa, Artificial Sequence
  Synthetic Peptide
RYRSCKDSFVFQCGFMP

SEQ ID NO: 417 - protein, 17 aa, Artificial Sequence
  Synthetic Peptide
GVTHCKDSFVYQCISGS

SEQ ID NO: 418 - protein, 17 aa, Artificial Sequence
  Synthetic Peptide
SPERCSDSFVYQCTSQR

SEQ ID NO: 419 - protein, 17 aa, Artificial Sequence
  Synthetic Peptide
PLSRCIDSFVYQCVSSS

SEQ ID NO: 420 - protein, 17 aa, Artificial Sequence
  Synthetic Peptide
LAGRCQDSFVFQCVEPT

SEQ ID NO: 421 - protein, 17 aa, Artificial Sequence
  Synthetic Peptide
AGTRCLDSFVFQCVAVG

SEQ ID NO: 422 - protein, 17 aa, Artificial Sequence
  Synthetic Peptide
VTDRCYDTFVFQCARGS

SEQ ID NO: 423 - protein, 17 aa, Artificial Sequence
  Synthetic Peptide
DHSRCQDTFVFQCGSRE

SEQ ID NO: 424 - protein, 17 aa, Artificial Sequence
  Synthetic Peptide
LVSSEADCFVYQCLSTR

SEQ ID NO: 425 - protein, 17 aa, Artificial Sequence
  Synthetic Peptide
SGRSGTDCFVYQCNGVD

SEQ ID NO: 426 - protein, 17 aa, Artificial Sequence
  Synthetic Peptide
SSGSETDCFVYQCGVVL

SEQ ID NO: 427 - protein, 17 aa, Artificial Sequence
  Synthetic Peptide
SSRSSTDCFVFQCVEQG

SEQ ID NO: 428 - protein, 17 aa, Artificial Sequence
  Synthetic Peptide
VLPTSGDCFVYQCDLTM

SEQ ID NO: 429 - protein, 17 aa, Artificial Sequence
  Synthetic Peptide
RTGRDRDCFVFQCHLSF

SEQ ID NO: 430 - protein, 17 aa, Artificial Sequence
  Synthetic Peptide
GLAQRVDCFVYQCQRGE

SEQ ID NO: 431 - protein, 15 aa, Artificial Sequence
  Synthetic Peptide
CIERDSFVYQACGRY

SEQ ID NO: 432 - protein, 15 aa, Artificial Sequence
  Synthetic Peptide
VCGTDSFVFQCGNEW

SEQ ID NO: 433 - protein, 15 aa, Artificial Sequence
  Synthetic Peptide
VRCADSFVFQQCSYP

SEQ ID NO: 434 - protein, 15 aa, Artificial Sequence
  Synthetic Peptide
YSCRDTFVFQCSVVR

SEQ ID NO: 435 - protein, 15 aa, Artificial Sequence
  Synthetic Peptide
SHCNDTFVFQCASSL

SEQ ID NO: 436 - protein, 15 aa, Artificial Sequence
  Synthetic Peptide
CRIRDAFVFQSCSRG

SEQ ID NO: 437 - protein, 15 aa, Artificial Sequence
  Synthetic Peptide
CVSRDAFVYQGCLDT

SEQ ID NO: 438 - protein, 15 aa, Artificial Sequence
  Synthetic Peptide
CSGSDAFVYQCHAHG

SEQ ID NO: 439 - protein, 15 aa, Artificial Sequence
  Synthetic Peptide
PSCGDAFVFQCGSRM

SEQ ID NO: 440 - protein, 15 aa, Artificial Sequence
  Synthetic Peptide
ACRLDTFVYQHGDVC

SEQ ID NO: 441 - protein, 15 aa, Artificial Sequence
  Synthetic Peptide
QRCIDTFVFQCSVSA

SEQ ID NO: 442 - protein, 15 aa, Artificial Sequence
  Synthetic Peptide
QRCIDTFVFQCSVSA

SEQ ID NO: 443 - protein, 15 aa, Artificial Sequence
  Synthetic Peptide
RRCMDSFVFQCRAAS

SEQ ID NO: 444 - protein, 15 aa, Artificial Sequence
  Synthetic Peptide
QGCMDTFVYQCKSSL

SEQ ID NO: 445 - protein, 15 aa, Artificial Sequence
  Synthetic Peptide
LGCMDSFVYQCGSFM

SEQ ID NO: 446 - protein, 15 aa, Artificial Sequence
  Synthetic Peptide
RPCEDSFVFQCGRYT

SEQ ID NO: 447 - protein, 15 aa, Artificial Sequence
  Synthetic Peptide
HGCEDTFVYQCGNAR

SEQ ID NO: 448 - protein, 15 aa, Artificial Sequence
  Synthetic Peptide
SSCEDTFVYQCITPV

SEQ ID NO: 449 - protein, 14 aa, Artificial Sequence
  Synthetic Peptide
QCGRDSFVFQCDYV

SEQ ID NO: 450 - protein, 15 aa, Artificial Sequence
  Synthetic Peptide
SCGPDSFVYQCWSPH

SEQ ID NO: 451 - protein, 15 aa, Artificial Sequence
  Synthetic Peptide
CHRYDTFVYQCGSTT

SEQ ID NO: 452 - protein, 15 aa, Artificial Sequence
  Synthetic Peptide
QCNVDSFVYQCWPRV

SEQ ID NO: 453 - protein, 15 aa, Artificial Sequence
  Synthetic Peptide
CLTLDTFVYQCSRSK

SEQ ID NO: 454 - protein, 15 aa, Artificial Sequence
  Synthetic Peptide
YTCLDTFVFQNEKCA

SEQ ID NO: 455 - protein, 15 aa, Artificial Sequence
  Synthetic Peptide
RCWMDSFVYQRCNTV

SEQ ID NO: 456 - protein, 15 aa, Artificial Sequence
  Synthetic Peptide
GVFIDSFVYQEVMSY

SEQ ID NO: 457 - protein, 14 aa, Artificial Sequence
  Synthetic Peptide
SASVDSFVYQGGRD

SEQ ID NO: 458 - protein, 15 aa, Artificial Sequence
  Synthetic Peptide
VQAYDSFVFQHVGYS

SEQ ID NO: 459 - protein, 15 aa, Artificial Sequence
  Synthetic Peptide
GREADSFVYQRGGGM

SEQ ID NO: 460 - protein, 15 aa, Artificial Sequence
  Synthetic Peptide
QSGRDSFVYQSGNGS

SEQ ID NO: 461 - protein, 12 aa, Artificial Sequence
  Synthetic Peptide
HSRFDTFVFQGH

SEQ ID NO: 462 - protein, 12 aa, Artificial Sequence
  Synthetic Peptide
HSRFDTFVFQGH

SEQ ID NO: 463 - protein, 15 aa, Artificial Sequence
  Synthetic Peptide
RPGLDTFVYQGTTSW

SEQ ID NO: 464 - protein, 15 aa, Artificial Sequence
  Synthetic Peptide
GGGLDSFVYQSTFTA

SEQ ID NO: 465 - protein, 15 aa, Artificial Sequence
  Synthetic Peptide
SGLIDSFVYQRELKE

SEQ ID NO: 466 - protein, 15 aa, Artificial Sequence
  Synthetic Peptide
RIEIDSFVYQGIVGR

SEQ ID NO: 467 - protein, 15 aa, Artificial Sequence
  Synthetic Peptide
RAIADSFVYQGGDLM

SEQ ID NO: 468 - protein, 12 aa, Artificial Sequence
  Synthetic Peptide
VEMSDTFVFQSL

SEQ ID NO: 469 - protein, 15 aa, Artificial Sequence
  Synthetic Peptide
GMITDAFVYQSHTGM

SEQ ID NO: 470 - protein, 15 aa, Artificial Sequence
  Synthetic Peptide
RSRSDSFVYQESVIH

SEQ ID NO: 471 - protein, 15 aa, Artificial Sequence
  Synthetic Peptide
GHVPDSFVFQTFGGE

SEQ ID NO: 472 - protein, 6 aa, Artificial Sequence
  Synthetic Peptide
DTFVFQ

SEQ ID NO: 473 - protein, 6 aa, Artificial Sequence
  Synthetic Peptide
DTFVYQ

SEQ ID NO: 474 - protein, 6 aa, Artificial Sequence
  Synthetic Peptide
DSFVFQ

SEQ ID NO: 475 - protein, 6 aa, Artificial Sequence
  Synthetic Peptide
DCFVYQ

(19) United States
(12) Patent Application Publication
(10) Pub. No.: US 2016/0033528 A1
(43) Pub. Date: Feb. 4, 2016

(54) Methods for Selecting Peptides that Bind to Disease Specific Antibodies, Disease Specific Peptides and Uses Thereof

(71) Applicant: The Regents of the University of California, Oakland, CA (US)

(72) Inventors: Patrick S. Daugherty, Santa Barbara, CA (US); John T. Ballew, San Antonio, TX (US)

(73) Assignee: The Regents of the University of California

(21) Appl. No.: 14/595,909

(22) Filed: Jan. 13, 2015

Related U.S. Application Data
(60) Provisional application No. 61/926,768, filed on Jan. 13, 2014.

(51) Int. Cl. G01N 33/68; C07K 7/06; C07K 7/08

(52) U.S. Cl. CPC G01N 33/6854; C07K 7/08; C07K 7/06

(57) ABSTRACT

Provided herein is a method for selecting and expanding polypeptide epitopes against disease-specific antibodies present in blood across a wide variety of antibody-mediated infectious and autoimmune diseases. In particular, high-throughput selection methods are provided for selecting and expanding disease-relevant polypeptide epitopes against disease-specific antibodies present in a sample from a subject using polypeptide epitope libraries. Also provided are polypeptide epitope sequences, which accurately discriminate subjects with active and remitting Celiac Disease compared to non-Celiac subjects for diagnostic or therapeutic purposes. These peptide epitopes can be employed in methods for diagnosing a subject with Celiac disease. Kits useful in the disclosed methods are also provided.

### CROSS-REFERENCE TO RELATED APPLICATION

This application claims the benefit of U.S. Provisional Application No. 61/926,768, filed Jan. 13, 2014, which is herein incorporated in its entirety.

### STATEMENT REGARDING FEDERALLY SPONSORED RESEARCH OR DEVELOPMENT

This invention was made with Government Support under AI090224 by the National Institutes of Health. The Government has certain rights in the invention.

### REFERENCE TO SEQUENCE LISTING

The Sequence Listing submitted May 22, 2015 as a text file named “UCSB_{—}2013_{—}828_Sequence_Listing.txt”, created on May 5, 2015, and having a size of 135,860 bytes is hereby incorporated by reference pursuant to 37 C.F.R. §1.52(e)(5).

### FIELD OF THE INVENTION

The invention relates epitopes for diagnosing disease and to methods for identifying such.

### BACKGROUND OF THE INVENTION

The diagnosis of many diseases relies heavily upon the accuracy of antibody detection. Assays to detect antibodies using known antigens are used extensively to diagnose infectious and autoimmune diseases.

The circulating antibody repertoire provides a rich source of potential diagnostic information. Serum antibodies serve as one of the largest classes of clinical disease biomarkers (Anderson, et al., J. Proteome Res., 4:1123-1133 (2005); Schellekens, et al., J. Arthritis Rheum., 43:155-163 (2000); Dieterich, et al., Nat. Med., 3:797-801 (1997)) owing to their intrinsic affinity and specificity, analytical stability, and amplification by the immune system. Thus, antibody detection assays can enable rapid and accurate diagnosis of diseases with high sensitivity and specificity and are amenable to point-of-care diagnostic applications. Unique antibody reactivity patterns or signatures, generated by multiple distinct antibody specificities, have been observed in many diseases including cancer (Wang, et al., N. Engl. J. Med., 353:1224-1235 (2005)); Anderson, et al., J. Proteome Res. 10(1):85-96 (2011)), autoimmune (Binder, et al., Autoimmunity Rev., 5:234-241 (2006), Anderson, et al. J. Proteome Res. 10(1):85-96 (2011)), neurodegenerative (Nagele, et al., PLoS One, 6,:e23112 (2011)), neurological and psychiatric disorders (Reddy, et al., Cell 144(1):132-142 (2011)) and infectious diseases (Kouzmitcheva, et al., Clin. Diagn. Lab. Immunol., 8:150-160 (2001)). Additionally, since some disease associated antibodies play a role in pathology (Caja, et al., Cell. Mol. Immunol., 8:103-109 (2011) monitoring their concentration in blood can provide valuable information to guide therapy (Kagnoff, J. Clin. Invest., 117:41-49 (2007); Brennan, et al., Nat. Rev. Cancer, 10:605-617 (2010)).

[symbol]

The utility of antibodies in diagnostics derives from their intrinsic affinity and specificity, biochemical stability, and abundance in blood. Nevertheless, the identification of rare antibody specificities indicative of disease and the development of reagents for their accurate detection have proven exceptionally difficult (Fritzler, Autoimmun Rev 7(8):616-620 (2011)). Inter-subject variability of antibody specificities is a major challenge to the development of accurate tests. Specifically, individual genetic and stochastic variations that shape the antibody repertoire introduce heterogeneity in disease antibody subpopulations (polyclonal variation, specificity, affinity, and titer), which hinders uniform antibody detection (Shere, et al., Semin Arthritis Rheum. 34(2):501-537 (2004) and Huizinga, et al., Arthritis Rheum. 52(11):3433-3438 (2005)).

The identification of serum antibody specificities that indicate disease and reagents for their detection has proven remarkably challenging.

Random peptide libraries (RPLs) have been proposed as a potential source of diagnostic reagents capable of mimicking diverse biological antigens in the environment (Cortese, et al., Trends Biotechnol. 12(7):262-267 (1994); Kouzmitcheva, et al., Clin. Diagn. Lab Immunol. 8(1):150-160 (2001); and Bartoli, et al., Nat. Biotechnol. 16(11):1068-1073 (1998)). Individual peptides identified from RPLs using patient sera have been capable of identifying patients with disease with modest accuracy (Bartoli, et al., Nat. Biotechnol. 16(11)10:1068-1073 (1998); Osman, et al., Clin. Exp. Immunol. 121(2):248-254 (2000)). Diagnostic accuracy can be improved in some cases using panels of library-isolated peptides coupled with statistical classification algorithms (Spatola, et al., Anal Chem., 85(2):1215-22 (2013)), with the drawback of requiring multiple independent measurements. In spite of these advances, peptides identified from random libraries have exhibited insufficient diagnostic efficacy (sensitivity and specificity) to foster their clinical development (Spatola, et al., Anal. Chem., 85(2):1215-22 (2013); Cortese, et al., Proc. Natl. Acad. Sci. USA 93(20):11063-11067 (1996); and Zanoni, et al., PLoS Med. 3(9):e358 (2006)).

While approved antibody-based diagnostic assays often exhibit sensitivity and/or specificity values in excess of 95% (Leffler, et al., Am. J. Gastroenterol. 105(12):2520-2524 (2010); van Venrooij, et al., Nat. Rev. Rheumatol. 7(7):391-398 (2011)), library isolated peptides that mimic antigens (mimotopes), used alone or in combination rarely meet these stringent requirements. For example, peptides from RPLs selected against serum antibodies from patients with Crohn's disease (Saito, et al., Gut, 52(4):535-540 (2003), multiple sclerosis (Cortese, et al., Proc. Natl. Acad. Sci, USA 93(20):11063-11067 (1996); Cortese, et al., Mult. Scler., 4(1):31-36 (1998); Fujimori, et al., Multiple Sclerosis International 2011:353417 (2011)), celiac disease (Spatola, et al.,) Anal. Chem., 85(2):1215-22 (2013); Zanoni, et al., PLoS Med., 3(9):e358 (2006)), rheumatoid arthritis (Dybwad, et al., Clin. Immunol. Immunopathol. 75(1):45-50 (1995)), or type-1 diabetes (Mennuni, et al., Journal of Molecular Biology 268(3):599-606 (1997); Mennuni, et al., J. Autoimmun. 9(3):431-436 (1996); and Bason, et al., PLoS One 8(2):e57729-e57729 (2013)), have exhibited insufficient diagnostic accuracy.

Consequently, there remains a need for discovery processes to identify antibody detection reagents exhibiting accuracies desired for clinical development. There is therefore a need for methods to identify disease specific epitopes with improved diagnostic efficacy (sensitivity and specificity). In addition, although antibody profiling methods (including phage and bacterial display) using RPLs lend themselves to various in vitro directed evolution protocols, this capability has not been exploited using blood specimens from patients.

It is therefore an object of the present invention to provide a method for identifying disease specific epitopes with improved sensitivity and specificity.

It is also an object of the invention to provide disease specific epitopes for identifying subjects with a specific disease.

It is still a further object of the invention to provide a method for diagnosing subjects with disease.

It is a further object of the invention to provide kit useful for diagnosing subjects with disease.

### SUMMARY OF THE INVENTION

Provided herein is a method for identifying disease specific epitopes with improved sensitivity and/or specificity in fluid specimens from subjects, preferably, human subjects. Preferably, the disease is celiac disease.

The method includes a first step of contacting diluted fluid specimen from disease subjects with a random peptide library, and then contacting the resulting sample with an antibody binding reporter protein. The random peptide library is displayed preferably on a bacterial cell, for example, E. coli. The method includes as a second step, measuring antibody binding to the peptide library, and recovering cells that bind to antibodies in the fluid specimen. The library-displaying cells recovered from the second step are contacted with pooled diluted specimen from one or more subjects without disease (control). Library displaying cells that do not bind to control subjects' antibodies are recovered (subtracted out) and the peptide sequences (which preferentially bind to antibodies from disease specimen) are identified (containing a core motif).

In a preferred embodiment, the method further includes constructing a focused peptide library from the peptide sequences (i.e., the peptides containing the core motif) identified from the random library, and repeating the steps of (i) contacting diluted pooled fluid specimen from disease subjects, (ii) identifying peptide displaying cells that bind to antibodies in disease specimen, (iii) subtracting out cells that do not bind to diluted pooled fluid specimen from control subjects, and (iv) identifying the disease-specific peptides. This process may be repeated two or more times as described below, each time, using: (a) a different set of disease and control subjects, and (b) a more focused peptide library, constructed using a consensus disease specific consensus sequence identified from the previous step. The process steps can be repeated 2-6 times, to identify disease-specific epitopes with diagnostically relevant specificity and sensitivity.

Each diluted specimen preferably includes specimen pooled from 2 to 10 subjects, in some embodiments, more preferably, pooled from 2 to 5 subjects. Preferably, the random peptide library is presented on the surface of bacterial cells. The fluid specimen may be any specimen that contains antibodies, for example, serum, plasma, cerebrospinal fluid (CSF), saliva, or urine. The fluid specimen is preferably serum. The fluid specimen is preferably diluted to a ratio ranging from 1:100 and 1:200. The antibody binding reporter protein is preferably a labeled antibody against immunoglobulin (Ig) IgG, IgA or IgM.

Also provided are epitopes for diagnosing subjects with a specific disease or condition. Preferably, the patient has celiac disease, and the epitopes are celiac-specific epitopes (CSE) and show improved specificity for celiac disease (CD). Antibodies to the CSE, and methods for using the CSE and anti-CSE antibodies are provided. The CSE epitope includes the peptide sequence CXD^{S}/_{T}FV^{Y}/_{F}QC (SEQ ID NO: 1). In a preferred embodiment, the CSE epitope is

(SEQ ID NO: 2)
MDVRCRDSFVYQCHVGT
or
(SEQ ID NO: 38)
QRCIDTFVFQCSVSA.

The CSE can be used in a method for diagnosing patients with celiac disease, by contacting a fluid specimen from a subject and identifying interaction of antibodies in the specimen with the CSE described herein. CSE antibody detecting peptides (SEQ ID NOs: 1 and 2) are useful in a method for diagnosing patients with the anti-CSE subtype of CD.

Peptides useful for diagnosis of CD with improved sensitivity are also provided.

In another embodiment, the celiac disease specific peptides may include the sequences FPEQPFPE (SEQ ID NO: 3), QPEQAFPE (SEQ ID NO:4) or expanded epitope P(^{P}/_{R}/^{M})EPQPEQPFPE (SEQ ID NO:5). In this embodiment, the peptide can include PPEPQPEQAFPE (SEQ ID NO: 6), PREPQPEQAFPE (SEQ ID NO: 7), or PMEPQPEQPFPE (SEQ ID NO: 8). In still other embodiments, the peptide includes DGP3 (RGRAQPEQAFPESVG) (SEQ ID NO: 9).

A kit useful in the methods described is preferably an ELISA kit. The kit includes the peptide epitopes disclosed herein, non-naturally occurring fusion proteins containing the peptide epitopes disclosed herein and secondary anti-IgG or IgA antibodies. For example, the kit can contain a fusion peptide containing the peptide QNGIDMFVYQGALA (SEQ ID NO: 39) or an equivalent peptide substituted with one or more conservative amino acid substitutions.

### BRIEF DESCRIPTION OF THE DRAWINGS

FIGS. 1A-1D show quantitative library screening methods used to identify and evolve antibody detecting peptides. FIG. 1A is an illustrated flow chart of the steps performed when a bacterial display random peptide library is subjected to repeated cycles of enrichment and subtraction with a sequence of pooled sera from CD and non-CD groups. FIG. 1B is an illustrated flow chart which shows flow cytometry enabled bacterial display peptide library screening for binders to CD and non-CD group serum antibodies. FIGS. 1C and 1D show measurement of immunoglobulin reactivity with the enriched library pools by class from the X₁₅ (FIG. 1C) or X₄CX₇CX₄ (FIG. 1D) with all CD sera groups and none of the non-CD groups. Each data point in FIGS. 1C and 1D represents a unique group of pooled sera (n=8 patients per pool).

FIGS. 2A-2C show directed evolution of antibody detecting peptides which increases their sensitivity and specificity. FIGS. 2A and C show evolved consensus epitopes for PEQ motif (Figure (A) and CSE (FIG. 2C) generated using WebLOGO3.0. FIG. 2B is a box-and-whiskers plot of the reactivity (fluorescence intensity) of each CD and non-CD sera group of bacterial clones expressing the PEQ-related peptides in (Table 4A, upper panel) pooled and assessed for IgG reactivity to five CD and five non-CD sera groups. The median value is plotted as a line with each box displaying the distribution of the inner quartiles with whiskers showing the upper and lower quartiles (all differences are statistically significant, p<0.0001).

FIGS. 3A and 3B show measurement of blinded patient sera (n=78) for IgG reactivity using DGP3 and DGP6 (FIG. 3A) or Quanta Lite™ (FIG. 3B). FIG. 3C shows assay results using ADEPt DGP3 epitope correlate with those obtained using Quanta Lite™ (Spearman's coefficient ρ=0.89). FIG. 3D shows Serum IgA antibody reactivity to DSFVYQ (SEQ ID NO: 3) epitope in 231 patient samples. FIG. 3E shows reactivity to DSFVYQ (SEQ ID NO: 3) in matched sera from CD patients before and after one year of GFD (gluten free diet). FIG. 3F shows mean fluorescence intensity for DGP3 and expanded epitope in CD subjects.

FIGS. 4A-4C show mimicry assays of celiac-specific epitope deamidated gamma-like epitope derived from library sorting (FIG. 4A), the deamidated alpha-gliadin epitope (FIG. 4B) or epitopes of open conformation TG2 (FIG. 4C) as tested by ELISA.

FIGS. 5A-5B show proteins and organisms identified by query of QPEQAFPE (SEQ ID NO:4) (FIG. 5A) and PFPEQXFP (SEQ ID NO:6) (FIG. 5B) against the non-redundant protein database using BLASTp (PAM30 Matrix) and rank ordered by total score.

### DETAILED DESCRIPTION OF THE INVENTION

A method for selecting and expanding peptide epitopes against disease-specific antibodies present in a sample across a wide variety of antibody-mediated infectious and autoimmune diseases is described. In particular, high-throughput selection methods are provided for selecting and expanding disease-relevant polypeptide epitopes against disease-specific antibodies present in human blood serum using polypeptide epitope libraries fused on the surface of engineered bacterial cells. More particularly, peptide epitope sequences that are able to accurately discriminate human subjects with active and refractory celiac disease compared to non-celiac humans, for diagnostic or therapeutic purposes are provided.

### I. Definitions

“Immobilization” as used herein refers to any coupling, binding or other association between the disclosed peptides and the support that prevents separate movement of the peptide and the support.

As used herein, “specifically binds” refers to the character of a receptor which recognizes and interacts with a ligand but does not substantially recognize and interact with other molecules in a sample, under given conditions.

### II. Method for Identifying Disease Specific Epitopes

The method described herein can be used to identify peptides specific for a specific disease/disorder that show improved specificity over prior art epitopes.

The results described herein demonstrate that in vitro directed evolution can be applied for de novo generation of reagents that exhibit requisite levels of diagnostic sensitivity and specificity for clinical translation. The results also show that in vitro evolution of such diagnostic reagents may provide a route to identify previously unknown environmental antigens involved in disease, and thereby elucidate pathobiology mechanisms.

A. Celiac Disease (CD)

# CD is an autoimmune, digestive disorder in which the small intestine (the part of the gut that absorbs nutrients from food) is damaged. Genetically susceptible individuals develop autoimmune injury to the gut, skin, joints, liver, brain, heart, uterus, and other organs. In addition, celiac patients show an increased prevalence of other autoimmune diseases. CD is considered a model for autoimmune disorders because the crucial genetic and environmental factors responsible for its pathogenesis have been identified. It is well known that the interplay of four components induces enteropathy: gluten/gliadin, gluten-specific T cells, the major histocompatibility complex locus HLA-DQ, and the endogenous enzyme tissue transglutaminase (tTG) (Hadjivassiliou, et al., Trends Immunol., 25:578-582 (2004)). Patients with active CD have immunoglobulin (Ig)A and IgG antibodies directed against tTG.

Giovanna, et al., PLOS Med., 3(9):e358 (2006) disclose a study using purified antibodies from blood provided by patients with CD to identify celiac peptide, a synthetic peptide VVKGGSSSLGW (SEQ ID NO: 10), that was specifically recognized by serum IgA of 68% individual patients with CD.

Spatola, et al., Anal. Chem. (2013) disclose the use of bacterial display random peptide libraries and purified antibody fractions, to screen for peptides that capture CD patient antibodies. However, the diagnostic efficiency of the peptide panel was insufficient for clinical translation and this methodology could not identify immunodominant epitopes or antigens involved in CD. The peptides identified in Spatola et al., when combined together in a panel, identified CD patients with 85% sensitivity and 91% specificity (as estimated using the statistical technique known as leave-one-out cross validation). This level of specificity is insufficient for clinical use because it would misdiagnose at least 1 in 10 patients, leading to an unnecessary biopsy and/or a lifelong gluten free diet.

B. Identifying Disease-Specific Epitopes

By identifying the appropriate assay sample type, assay sample size and quantitative screening algorithm, the methods disclosed herein enable precise identification of immunodominant B-cell epitopes involved in disease. The screening algorithm utilizes sequential library enrichment and depletion against a series of disease and non disease sera, for example, CD and non-CD pooled sera, to enrich peptides that specifically bind antibodies from disease subjects.

The method includes sorting a polypeptide cell surface display library, for example, (i) using a bacterial cell surface polypeptide display library to isolate peptides reactive to antibodies present in pooled fluid specimen derived from subjects with disease, (ii) counter-screening to remove library member peptides non-specific to disease, and (iii) design and iterative selection of generational libraries for expansion of polypeptide motifs derived from prior library cycles.

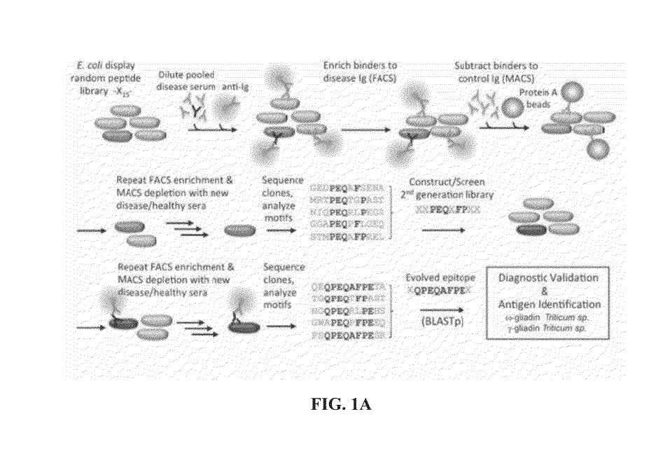

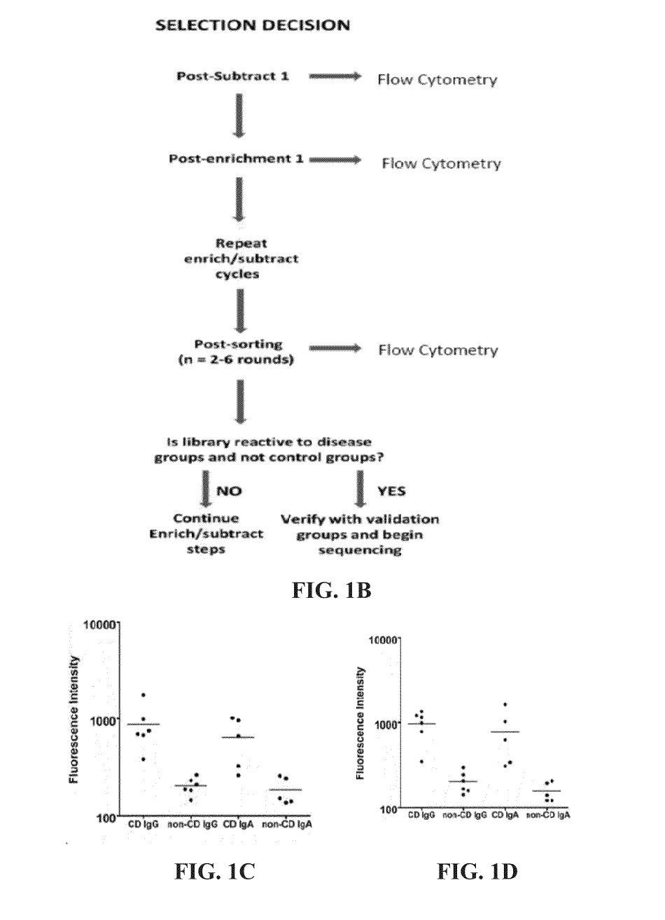

The method for identifying disease-specific peptides that bind antibodies from subjects with disease is depicted in FIG. 1A. The method includes contacting a random peptide library with fluid specimen from one or more subjects with disease. The peptide library is preferably a bacterial cell peptide library.

The sample (i.e., fluid specimen+peptide library) is washed, and then contacted with an antibody binding reporter protein. In a preferred embodiment, the antibody binding reporter peptide is a labeled antibody against immunoglobulin (Ig) G, IgA or IgM. In another embodiment, the antibody binding reporter protein is a labeled protein A or protein G conjugate.

Antibody binding to bacterial cells is then measured and bacterial cells that bind to antibodies in fluid specimen from the disease subject are recovered. Identification of antibody-random peptide library interaction and recovery of cells that bind disease subject antibodies described above is performed using methods known in the art, preferably, fluorescence-activated cell sorting (FACS), or magnetic-activated cell sorting (MACS).

The recovered disease subject antibody-binding cells are then contacted with pooled diluted specimen from one or more subjects without disease (control). Peptide library displaying cells that do not bind to the control subjects' antibodies are also recovered using methods known in the art, preferably, magnetic cell sorting (MACS) or FACS. See for example, FIG. 1A.

The method further includes constructing a second (focused) peptide library encompassing the consensus sequence identified from the random peptide library, in which one or more sequence positions are fixed to amino acids within the consensus sequence or biased toward amino acids within the consensus sequence. The focused library is contacted with diluted sample from subjects with disease, and then with an antibody binding protein reporter as described above. The disease subject sample group is not the same sample group used with the random protein library. Cells that bind to disease subject antibodies are recovered. The recovered cells are contacted with diluted specimen from subjects without disease (control), and cells that do not bind to control subject derived serum antibodies are recovered. The control subjects used to subtract cells displaying the focused library, that bind to disease subject antibody, are not the same control subjects used to subtract cells displaying the random peptide library, that bind to disease subject antibodies.

These steps identify one or more consensus sequence motifs among peptides binding to disease subject antibodies but not to antibodies from subjects without disease. The consensus sequence thus identified can be used to construct a second focused library, which is screened as described above, in order to identify disease specific epitopes with increased specificity and sensitivity. The disease subject and control subject specimen is diluted as describe above, preferably however, the specimen is diluted to a ratio between 1:500 and 1:1000. In some embodiments, the specimen is diluted to a ration between 1:100 and 1:200.

After each round of subtraction and enrichment, specificity can quantified by measuring library population reactivity to multiple CD and non-CD groups using flow cytometry, for example. FIG. 1B. After 2-6 subtraction/enrichment rounds, enriched libraries exhibiting high specificity for patient pools can be identified.

These process steps are repeated two or more times as described below, each time, using a different set of disease subjects and control subjects, and a more focused peptide library, constructed using a disease specific consensus sequence identified from the previous step.

1. Fluid Specimens

The fluid specimen includes specimens that contain antibodies, for example serum, plasma, CSF, saliva, or urine. Preferably, the fluid specimen is human serum. The fluid specimen is obtained from 2 to 10 disease subjects and pooled. In some embodiments the fluid specimen is obtained front 2 to 8 disease subjects, preferably, from 2 to 5 disease subjects. The fluid specimen is preferably diluted to a ratio between 1:100 and 1:200.

2. Peptide Display Systems

The method described herein can employ any peptide display system known in the art. Numerous peptide display systems have been described in the art. For example, display of peptides on the surface of filamentous bacteriophage, or phage display, has proven a versatile and effective methodology for the isolation of peptide ligands binding to a diverse range of targets. Scott, et al. Science, 249(4967):386-904 (1990); Norris, et al., Science, 285(5428):744-765 (1999); Arap, et al., Science, 279(5349):377-806 (1998); and Whaley, et al., Nature, 405(6787):665-668 (2000). Polypeptide display systems include mRNA and ribosome display, eukaryotic virus display, and bacterial and yeast cell surface display. Phage display involves the localization of peptides as terminal fusions to the coat proteins, e.g., pIII, pIIV of bacteriophage particles (Scott, et al., Science 249(4967):386-390 (1990); and Lowman, et al., Biochem., 30(45):10832-10838 (1991)). Other display formats and methodologies include mRNA display, ribosome or polysome display, eukaryotic virus display, and bacterial, yeast, and mammalian cell surface display. Matthcakis, et al., PNAS USA, 91(19): 9022-9026 (1994); Wilson, et al., PNAS USA, 98(7):3750-3755 (2001); Shusta, et al. Curr. Opin. Biotech., 10(2):117-122 (1999); and Boder, et al., Nature Biotech., 15(6):553-557 (1997).

In a preferred embodiment, the peptide display system is a bacterial display system as described for example in U.S. Pat. No. 8,361,933, the contents of which are herein incorporated by reference.

### III. Peptides and Antibodies

The methods disclosed above were applied within the context of Celiac disease, leading to identification of peptide epitopes that can be used to diagnose a subject as having celiac disease. In some embodiments the celiac specific peptides can be used to raise antibodies, which themselves can find use in a method for identifying the presence of these peptides in different foods.

A. Celiac-Specific Peptides

Unique polypeptide epitope sequences are provided which i) demonstrate specific (100%) and sensitive (98%) discrimination of Celiac and non-Celiac patients in a one-way blind trial (n=78 human subjects), and ii) specifically detect recovering Celiac Disease subjects seronegative for classical CD antibody biomarkers used in tracking disease remission (e.g. anti-deamidated gliadin peptides, anti-transglutaminase 2). In still another embodiment, CSE epitopes are provided which demonstrate 71% sensitivity and 99% specificity in discriminating between of celiac and non-celiac patients. CSE epitopes are useful to serologically subtype patients with CD, and to detect CD while patients have been on a gluten free diet at which time TG2 and DGP antibody titers are typically insufficient for disease detection. In one embodiment, the CSE epitope includes the sequence CXD^{S}/_{T}FV^{Y}/_{F}QC (SEQ ID NO: 1) or P(^{P}/_{R}/^{M})EPQPEQPFPE (SEQ ID NO: 5). In a preferred embodiment, the CSE epitope includes the peptide DxFVYQ (SEQ ID NO: 11) or DxFVFQ (SEQ ID NO: 12). In these embodiments, the peptide can include the sequence IDxFVYQGA (SEQ ID NO: 13), where x is any amino acid or

(SEQ ID NO: 2)
MDVRCRDSFVYQCHVGT.

In other embodiments, the peptide includes the sequence QPEQAFPE (SEQ ID NO: 4) or PFPEQxFP (SEQ ID NO:29). Additional useful peptides with the PEQ motif are listed below.

TABLE 1A
PEQXFP S5E5 1:1000 SORTS

| | | | |
|---|---|---|---|
| G Q S G Q S V A G Q | SEQ ID NO: 40 | G Q S G Q K I Y S Q P E Q G F P | SEQ ID NO: 50 |
| P E Q A F P D R V M | | E K W M | |
| G Q S G Q G A R T Q | SEQ ID NO: 41 | G Q S G Q P E Q A F P D G L S | SEQ ID NO: 51 |
| P E Q A F P E E Y G | | (2X) | |
| G Q S G Q S L L A Q | SEQ ID NO: 42 | G Q S G Q R P G W Q P E Q A F | SEQ ID NO: 52 |
| P E Q G F P E Q W R | | P E G I R | |
| (2X) | | | |
| G Q S G Q R W T N Q | SEQ ID NO: 43 | G Q S G Q G G S S Q P E Q A F P | SEQ ID NO: 53 |
| P E Q A F P D R S S | | E Y R V | |
| G Q S G Q G G L G Q | SEQ ID NO: 44 | G Q S G Q S A W S Q P E Q S F P | SEQ ID NO: 54 |
| P E Q S F P E R V K | | E L G H (3X) | |
| G Q S G Q R R G A Q | SEQ ID NO: 45 | G Q S G Q R A G I Q P E Q S F P | SEQ ID NO: 55 |
| P E Q S F P D L G M | | E V G L | |
| G Q S G Q G S L A Q | SEQ ID NO: 46 | | |
| P E Q A F P D E K R | | | |
| G Q S G Q S V T A F | SEQ ID NO: 47 | G Q S G Q P Q T P F P E Q V F P | SEQ ID NO: 56 |
| P E Q A F P M I R G | | S L G Q | |
| G Q S G Q G W S A F | SEQ ID NO: 48 | G Q S G Q P A A P F P E Q E F P | SEQ ID NO: 57 |
| P E Q E F P L R Q Q | | T G H R | |
| G Q S G Q V S Q A F | SEQ ID NO: 49 | G Q S G Q V S V R G P E Q P F P | SEQ ID NO: 58 |
| P E Q E F P R I V K | | R L I S | |

TABLE 1B
PEQXFP S5E5 1:500 SORTS
Examples of peptides with the DxF motif (2^{nd} and third generation peptides)
are provided in the tables below.

| | | | |
|---|---|---|---|
| G Q S G Q V M N S Q P E | SEQ ID NO: 59 | G Q S G Q P Y R G S P E | SEQ ID NO: 76 |
| Q S F P D A R | | Q P F P V L E G | |
| G Q S G Q A G E N S P E | SEQ ID NO: 60 | G Q S G Q G Q R F M P E | SEQ ID NO: 77 |
| Q P F P L S | | Q A P P M I G M | |
| G Q S G Q R L G A G P E | SEQ ID NO: 61 | G Q S G Q P V V A F P E | SEQ ID NO: 78 |
| Q P F P V R S F | | Q L F P K L L K | |
| G Q S G Q V G P V L P E | SEQ ID NO: 62 | G Q S G Q E L R V Q P F | SEQ ID NO: 79 |
| Q S F P G W M G | | Q S F P E S L R | |
| G Q S G Q G F K L G P E | SEQ ID NO: 63 | G Q S G Q S T R S N P E | SEQ ID NO: 80 |
| Q R F P F M E V | | Q P F P S GVE | |
| G Q S G Q P E Q G F P S | SEQ ID NO: 64 | G Q S G Q S G W R L P E | SEQ ID NO: 81 |
| S W N | | Q A F P M Q M N | |
| G Q S G Q P V Q S F P E | SEQ ID NO: 65 | G Q S G Q S L E A F P E | SEQ ID NO: 82 |
| Q L F P R G G S | | Q G F P G P R E | |
| G Q S G Q P G I A F P E | SEQ ID NO: 66 | G Q S G Q R V A W T P E | SEQ ID NO: 83 |
| Q Q F P N L P G | | Q S F P F P E K | |
| G Q S G Q V M I K A P E | SEQ ID NO: 67 | G Q S G Q R A R I M P E | SEQ ID NO: 84 |
| Q A F P G R G L | | Q A F P G G L I | |
| G Q S G Q R Y R V Q P E | SEQ ID NO: 68 | G Q S G Q S G T G N P E | SEQ ID NO: 85 |
| Q G F P E G M A | | Q P F P Q L D R | |
| G Q S G Q G L E M S P E | SEQ ID NO: 69 | G Q S G Q G L M G Q P E | SEQ ID NO: 86 |
| Q G F P F R E R | | Q S F P E V V S | |
| G Q S G Q V L M E G P E | SEQ ID NO: 70 | G Q S G Q S V A G Q P E | SEQ ID NO: 87 |
| Q A F P G A A K | | Q A F P D R V M | |
| G Q S G Q K I Y S Q P E | SEQ ID NO: 71 | G Q S G Q G A R T Q P E | SEQ ID NO: 88 |
| Q G F P E K W M | | Q A F P E L Y G | |
| x x x x x G Q S G Q P E | SEQ ID NO: 72 | G Q S G Q S L L A Q P E | SEQ ID NO: 89 |
| A F P D G L S | | Q G F P E Q W R | |
| G Q S G Q R P G W Q P E | SEQ ID NO: 73 | G Q S G Q R W T N Q P E | SEQ ID NO: 90 |
| Q A F P E G I R | | Q A F P D R S S | |
| G Q S G Q G G S S Q P E | SEQ ID NO: 74 | G Q S G Q G G E G Q P E | SEQ ID NO: 91 |
| Q A F P E Y R V | | Q S F P E R V K | |
| G Q S G Q S A W S Q P E | SEQ ID NO: 75 | G Q S G Q R R G A Q P E | SEQ ID NO: 92 |
| Q S F P E L G H | | Q S F P D L G M | |
| G Q S G Q R A G I Q P E | SEQ ID NO: 93 | G Q S G Q G S L A Q P E | SEQ ID NO: 109 |
| Q S F P E V G L | | Q A F P D E K R | |
| G Q S G Q S V T A F P E | SEQ ID NO: 94 | G Q S G Q P Q T P F P E | SEQ ID NO: 110 |
| Q A F P M I R G | | Q V F P S L G Q | |
| G Q S G Q G W S A F P E | SEQ ID NO: 95 | G Q S G Q P A A P F P E | SEQ ID NO: 111 |
| Q E F P L R Q Q | | Q E F P T G H R | |
| G Q S G Q V S Q A F P E | SEQ ID NO: 96 | G Q S G Q V S V R G P E | SEQ ID NO: 112 |
| Q E F P R I V K | | Q P F P R L I S | |
| G Q S G Q V M N S Q P E | SEQ ID NO: 97 | G Q S G Q P Y R G S P E | SEQ ID NO: 113 |
| Q S F P D A R x | | Q P I F P V L E G | |
| G Q S G Q A G L V S P E | SEQ ID NO: 98 | G Q S G Q G Q R F M P E | SEQ ID NO: 114 |
| Q P F P L S x x | | Q A F P M I G M | |
| G Q S G Q R L G A G P E | SEQ ID NO: 99 | G Q S G Q P V V A F P E | SEQ ID NO: 115 |
| Q P F P V R S F | | Q L F P K L L K | |
| G Q S G Q V G P V L P E | SEQ ID NO: 100 | G Q S G Q E L R V Q P E | SEQ ID NO: 116 |
| Q S F P G W M G | | Q S F P E S E R | |
| G Q S G Q G F K L G P E | SEQ ID NO: 101 | G Q S G Q S T R S N P E | SEQ ID NO: 117 |
| Q R F P F M E V | | Q P I F P S G V L | |
| G Q S G Q P E Q G F P S | SEQ ID NO: 102 | G Q S G Q S G W R E P E | SEQ ID NO: 118 |
| S W N x x x x x | | Q A F P M Q M N | |
| G Q S G Q P V Q S F P E | SEQ ID NO: 103 | G Q S G Q S L E A F P E | SEQ ID NO: 119 |
| Q L F P R G G S | | Q G F P G P R E | |
| G Q S G Q P G I A F P E | SEQ ID NO: 104 | G Q S G Q R V A W T P E | SEQ ID NO: 120 |
| Q Q F P N L P G | | Q S F P F P E K | |
| G Q S G Q V M I K A P E | SEQ ID NO: 105 | G Q S G Q R A R I M P E | SEQ ID NO: 121 |
| Q A F P G R G L | | Q A F P G G L I | |
| G Q S G Q R Y R V Q P E | SEQ ID NO: 106 | G Q S G Q S G T G N P E | SEQ ID NO: 122 |
| Q G F P E G M A | | Q P F P Q L D R | |
| G Q S G Q G L L M S P E | SEQ ID NO: 107 | G Q S G Q G L M G Q P E | SEQ ID NO: 123 |
| Q G F P F R E R | | Q S F P E V V S | |
| G Q S G Q V L M E G P E | SEQ ID NO: 108 | | |
| Q A F P G A A K | | | |

TABLE 2A
S4E4 1:500 (3^{rd} generation)

| | | | |
|---|---|---|---|
| GHVRDSFVYQTFGGE | SEQ ID | YRSRDTFVYQNSSKR | SEQ ID |
| | NO: 124 | | NO: 136 |
| VDLRDTFVYQERSLR | SEQ ID | RLVRDSFVYQEKVYE | SEQ ID |
| | NO: 125 | | NO: 137 |
| SGLRDSFVYQEEGSS | SEQ ID | FPGRDTFVYQTGSQM | SEQ ID |
| | NO: 126 | | NO: 138 |
| IVMLDSFVYQDFKGR | SEQ ID | TLRLDTFVFQPGDDM | SEQ ID |
| | NO: 127 | | NO: 139 |
| GPVVDSFVYQTGSYI | SEQ ID | RGSVDSFVFQS | SEQ ID |
| | NO: 128 | | NO: 140 |
| SIVLDTFVFQGGQDR | SEQ ID | SLALDSFVYQGSRYQ | SEQ ID |
| | NO: 129 | | NO: 141 |
| VRPLDSFVYQQLSKE | SEQ ID | YGRQDSFVFRVLQLG | SEQ ID |
| | NO: 130 | | NO: 142 |
| MHITDSFVFQDGTNV | SEQ ID | SGTSDSFVFQEGENR | SEQ ID |
| | NO: 131 | | NO: 143 |
| RPESDSFVFQTEASA | SEQ ID | FIDADSFVYQRHTQL | SEQ ID |
| | NO: 132 | | NO: 144 |
| VIHADSFVFQGGLKE | SEQ ID | LSFMDTFVYQGTPAL | SEQ ID |
| | NO: 133 | | NO: 145 |
| PCASDTFVFQDRECG | SEQ ID | VRVEDTFVYQGRALL | SEQ ID |
| | NO: 134 | | NO: 146 |
| RLGFDSFVFQRYPKK | SEQ ID | | |
| | NO: 135 | | |

TABLE 2B
S4E4 1:1000 (3^{rd} generation)

| | | | |
|---|---|---|---|
| LVGLDSFVYQMGPVK | SEQ ID | VRLVDSFVFQGAAHL | SEQ ID |
| | NO: 147 | | NO: 158 |
| NTILDSFVYQGSVHP | SEQ ID | RNGLDTFVYQDAVVH | SEQ ID |
| | NO: 148 | | NO: 159 |
| HGRLDTYVYQPTLGR | SEQ ID | RYWLDSFVFQAPGCC | SEQ ID |
| | NO: 149 | | NO: 160 |
| LFQLDSFVYQGSLGW | SEQ ID | GPLRDTFVFQR | SEQ ID |
| | NO: 150 | | NO: 161 |
| PRWRDSFVYQHAMIE | SEQ ID | WACRDAFVYQDGCK | SEQ ID |
| | NO: 151 | | NO: 162 |
| CHVRDAFVYQGSCRL | SEQ ID | HTTRDSFVYQLEGER | SEQ ID |
| | NO: 152 | | NO: 163 |
| RQLRDSFVYQVVGN | SEQ ID | PRFRDSFVFQEQKLS | SEQ ID |
| | NO: 153 | | NO: 164 |
| YSGEDSFVFQRTGSG | SEQ ID | LIGDDTFVYQHVGQF | SEQ ID |
| | NO: 154 | | NO: 165 |
| KRCSDSFVFQRSSYC | SEQ ID | ARAADSFVFQYGPGE | SEQ ID |
| | NO: 155 | | NO: 166 |
| QCAEDSFVFQACSGG | SEQ ID | VDLSDSFVYQSLTRK | SEQ ID |
| | NO: 156 | | NO: 167 |
| VQAYDSFVFQHVGYS | SEQ ID | | |
| | NO: 157 | | |

TABLE 2C
S5E5 1:500 (3^{rd} generation)

| | | | |
|---|---|---|---|
| RCKNDSFVFQACSPH | SEQ. ID | CEGMDSFVYQCFSQY | SEQ. ID |
| | NO: 168 | | NO: 179 |
| SYCLDSFVYQSASCS | SEQ ID | SCIMDSFVYQGSQCL | SEQ ID |
| | NO: 169 | | NO: 180 |
| CDGIDTFVYQCSSNL | SEQ ID | CQTKDSFVYQCHYQE | SEQ ID |
| | NO: 170 | | NO: 181 |
| ALSKDTFVFQILAH | SEQ ID | HRDSFVFQYGGDG | SEQ ID |
| | NO: 171 | | NO: 182 |
| RIGLDSFVFQNPRVI | SEQ ID | YVELDTFVYQGNSSM | SEQ ID |
| | NO: 172 | | NO: 183 |
| NSAIDTFVFQSPTNP | SEQ ID | GAAVDSFVFQEPDS | SEQ ID |
| | NO: 173 | | NO: 184 |
| PRVMDSFVYQQVISL | SEQ ID | GSRMDSFVYQGFPWN | SEQ ID |
| | NO: 174 | | NO: 185 |
| ICESDTFVFQGSCRG | SEQ ID | GLGSDSFVYQSTSYT | SEQ ID |
| | NO: 175 | | NO: 186 |
| RESEDSFVYQQTRRV | SEQ ID | RMTDDSFVYQSLGRS | SEQ ID |
| | NO: 176 | | NO: 187 |
| RLVGDAFVFQRSSD | SEQ ID | SPLNDTFVYQRFLVP | SEQ ID |
| | NO: 177 | | NO: 188 |
| GTGTDSFVYQSTEPG | SEQ ID | MKRYDAFVFQDVQPL | SEQ ID |
| | NO: 178 | | NO: 189 |

TABLE 2D
S5E5 1:1000 (3^{rd} generation)

| | | | |
|---|---|---|---|
| GLCNDTFVYQDKCGS | SEQ ID | GLCVDTFVYQGQGCR | SEQ ID |
| | NO: 190 | | NO: 200 |
| HGCEDTFVYQCGNAR | SEQ ID | VECVDSFVYQCKRGS | SEQ ID |
| | NO: 191 | | NO: 201 |
| VRCVDSFVFQCTKRA | SEQ ID | SCGGDAFVYQCFVSS | SEQ ID |
| | NO: 192 | | NO: 202 |
| CHTKDAFVFQCDSNL | SEQ ID | TCMLDAFVFQTGLCG | SEQ ID |
| | NO: 193 | | NO: 203 |
| CFRADAFVYQGCDVM | SEQ ID | ASYRDSFVFQDHHTG | SEQ ID |
| | NO: 194 | | NO: 204 |
| IGMRDAFVYQIPNLH | SEQ ID | VERLDTFVYQRVDTR | SEQ ID |
| | NO: 195 | | NO: 205 |
| SLVLDSFVYQTGDRR | SEQ ID | LRQLDSFVYQGFEIV | SEQ ID |
| | NO: 196 | | NO: 206 |
| QGIIDSFVYQRSDPG | SEQ ID | LSAIDTFVFQSSPRE | SEQ ID |
| | NO: 197 | | NO: 207 |
| TQGSDSFVYQTMERG | SEQ ID | LRWHDTFVYQGSLSP | SEQ ID |
| | NO: 198 | | NO: 208 |
| ADTFVFQEMHVK | SEQ ID | | |
| | NO: 199 | | |

TABLE 3A
S4E4 (2^{nd} Generation)

| | | | |
|---|---|---|---|
| VDSGCVDSFVYQCRSLG | SEQ ID NO: 209 | RTGVCSDSFVYQCDPVA | SEQ ID NO: 220 |
| GFKKCSDSFVYQCMGGK | SEQ ID NO: 210 | GSAGCIDSFVFQCGLR | SEQ ID NO: 221 |
| LNGACGDSFVFQCTAGL | SEQ ID NO: 211 | RGDRCSDTFVFQCWTPD | SEQ ID NO: 222 |
| QLVHCYDTFVFQCDAAR | SEQ ID NO: 212 | GSRYGRDCFVFQCGVDL | SEQ ID NO: 223 |
| VSPLERDCFVYQCVS | SEQ ID NO: 213 | LFGLESDCFVYQCSNTP | SEQ ID NO: 224 |
| SPLMSGDCFVYQCATHS | SEQ ID NO: 214 | RLNSCRDLFVYQCWQAG | SEQ ID NO: 225 |
| AITCSHDAFVYQCGVPM | SEQ ID NO: 215 | LGTCGADPFVFQCQNIR | SEQ ID NO: 226 |
| SACGGVDNFVYQCSLAN | SEQ ID NO: 216 | GVCMARDAFVYQCLRGG | SEQ ID NO: 227 |
| VPGTCQDGFVYQCLWGA | SEQ ID NO: 217 | CSGLLMDRFVYQCDVVN | SEQ ID NO: 228 |
| GRACRSDAFVFQCGLSA | SEQ ID NO: 218 | CMSGVIDPFVFQCEGMG | SEQ ID NO: 229 |
| LPLTYFELFVFQCMCHM | SEQ ID NO: 219 | CSETFVFQCPMGA | SEQ ID NO: 230 |

TABLE 3B
S5E5 (2^{nd} Generation)

| | | | |
|---|---|---|---|
| GQSGQSSVLCRDTFVFQCTPVV | SEQ ID NO: 231 | GQSGQNRDRCRDSFVYQCVFPT | SEQ ID NO: 258 |
| GQSGQWRHGCADSFVFQCDAWQ | SEQ ID NO: 232 | GQSGQGTACHADSFVYQCSSQK | SEQ ID NO: 259 |
| GQSGQLRGGCADSFVYQCESSA | SEQ ID NO: 233 | GQSGQRSSFCSDSFVFQCELPG | SEQ ID NO: 260 |
| GQSGQVCEPFTDSFVYQCPSAN | SEQ ID NO: 234 | GQSGQRRKCSEDSFVYQCVRST | SEQ ID NO: 261 |
| (2x) | | | |
| GQSGQVSPLERDCFVYQCVSSS | SEQ ID NO: 235 | GQSGQSREGRGDCFVYQCHTSI | SEQ ID NO: 262 |
| GQSGQVLPTSGDCFVYQCDLTM | SEQ ID NO: 236 | GQSGQSSGSETDCFVYQCGVVL | SEQ ID NO: 263 |
| | | (2x) | |
| GQSGQVCQDEFVFQCSSA | SEQ ID NO: 237 | GQSGQRSAACSDEFVYQCRGNT | SEQ ID NO: 264 |
| (2x) | | | |
| GQSGQGCLDEFVFQCAGGS | SEQ ID NO: 238 | GQSGQMVRSCYDQFVYQCSQTS | SEQ ID NO: 265 |
| GQSGQGSGKCIDPFVYQCLRMS | SEQ ID NO: 239 | GQSGQGSGRCEDAFVYQCQSFG | SEQ ID NO: 266 |
| GQSGQLWCTRSDAFVYQCSRMQ | SEQ ID NO: 240 | GQSGQWAAQCVDGFVYQCGAHL | SEQ ID NO: 267 |
| GQSGQGMEAFPEQGFRSPA | SEQ ID NO: 241 | GQSGQMDVRCRDSTVYQCHVGT | SEQ ID NO: 268 |
| | | (3x) | |
| GQSGQRYRSCKDSFVFQCGFMP | SEQ ID NO: 242 | GQSGQGVTHCKDSFVYQCISGS | SEQ ID NO: 269 |
| GQSGQGCRDSFVFQCESGS | SEQ ID NO: 243 | GQSGQSKNDCRDSFVFQCGSRS | SEQ ID NO: 270 |
| GQSGQTVHGCRDSFVYQCESRG | SEQ ID NO: 244 | GQSGQSPERCSDSFVYQCTSQR | SEQ ID NO: 271 |
| GQSGQVTDRCYDTFVFQCARGS | SEQ ID NO: 245 | GQSGQDHSRCQDTFVFQCGSRE | SEQ ID NO: 272 |
| | | (2x) | |
| GQSGQPLSRCIDSFVYQCVSSS | SEQ ID NO: 246 | GQSGQLAGRCQDSFVFQCVEPT | SEQ ID NO: 273 |
| GQSGQAGTRCLDSFVFQCVAVG | SEQ ID NO: 247 | GQSGQVCEPFTDSFVYQCPSAN | SEQ ID NO: 274 |
| GQSGQPELQCFDSFVFQCNGGR | SEQ ID NO: 248 | GQSGQRGCNHMDTFVYQCPSG | SEQ ID NO: 275 |
| GQSGQLVSSEADCFVYQCLSTR | SEQ ID NO: 249 | GQSGQSGRSGTDCFVYQCNGVD | SEQ ID NO: 276 |
| | | (2x) | |
| GQSGQSSGSETDCFVYQCGVVL | SEQ ID NO: 250 | GQSGQSSRSSTDCFVFQCVEQG | SEQ ID NO: 277 |
| GQSGQVLPTSGDCFVYQCDLTM | SEQ ID NO: 251 | GQSGQRTGRDRDCFVFQCHLSF | SEQ ID NO: 278 |
| GQSGQGLAQRVDCFVYQCQRGE | SEQ ID NO: 252 | GQSGQCLDAFVYQCTANL | SEQ ID NO: 279 |
| GQSWERRRCLDAFVFQCVEK | SEQ ID NO: 253 | GQSGQGKSCHDDLFVYQCGSRM | SEQ ID NO: 280 |
| GQSGQALQACVDAFVYQCNSGS | SEQ ID NO: 254 | GQSGQPKRCHSDAFVFQCLPSG | SEQ ID NO: 281 |
| GQSGQSLHACYDAFVYQCNSGD | SEQ ID NO: 255 | GQSGQCQGLGDERFVFQCV | SEQ ID NO: 282 |
| GQSGQGCLDEFVFQCAGGS | SEQ ID NO: 256 | GQSGQIHVSCRDDFVYQCAYRQ | SEQ ID NO: 283 |
| GQSGQYGTACVDGFVYQCGVRG | SEQ ID NO: 257 | GQSGQPDQTCAEPFVYQCARGG | SEQ ID NO: 284 |

C. Isolated Antibodies

Also provided are isolated antibodies that bind CSE-specific peptides disclosed herein. The antibodies can be polyclonal or monoclonal antibodies. Starting from a particular peptide, a person skilled in the art is able, without undue effort, to generate an isolated antibody that specifically binds to the peptide. Such techniques and approaches are well-known to those skilled in the art and routine in daily laboratory practice.

A monoclonal antibody composition is typically composed of antibodies produced by clones of a single cell called a hybridoma that secretes (produces) only one type of antibody molecule. The hybridoma cell is formed by fusing an antibody-producing cell and a myeloma or other self-perpetuating cell line. Such antibodies were first described by Kohler and Milstein, Nature, 256:495-497 (1975), the disclosure of which is herein incorporated by reference. An exemplary hybridoma technology is described by Niman et al., Proc. Natl. Acad. Sci. U.S.A., 80:4949-4953 (1983). Other methods of producing monoclonal antibodies, a hybridorna cell, or a hybridoma cell culture are also well known. See e.g., Antibodies: A Laboratory Manual, Harlow et al., Cold Spring Harbor Laboratory, 1988; or the method of isolating monoclonal antibodies from an immunological repertoire as described by Sasatry, et al., Proc. Natl. Acad. Sci. USA, 86:5728-5732 (1989); and Huse et al., Science, 246:1275-1281 (1981). The references cited are hereby incorporated herein by reference.

In order to produce monoclonal antibodies, a host mammal is inoculated with the disclosed CSE and then boosted. Spleens are collected from inoculated mammals a few days after the final boost. Cell suspensions from the spleens are fused with a tumor cell in accordance with the general method described by Kohler and Milstein (Nature, 256:495-497 (1975). If the fragment is too short to be immunogenic, it may be conjugated to a carrier molecule. Some suitable carrier molecules include keyhole limpet hemocyanin and bovine serum albumin. Conjugation may be carried out by methods known in the art. One such method is to combine a cysteine residue of the fragment with a cysteine residue on the carrier molecule. The peptide fragments may be synthesized by methods known in the art. Some suitable methods are described by Stuart and Young in “Solid Phase Peptide Synthesis,” Second Edition, Pierce Chemical Company (1984).

The disclosed peptides may be utilized to prepare antibodies, monoclonal or polyclonal antibodies, and immunologically active fragments (e.g., a Fab or (Fab)₂ fragment), an antibody heavy chain, an antibody light chain, humanized antibodies, a genetically engineered single chain Fᵥ molecule (Ladne et al., U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Purification of the antibodies or fragments can be accomplished by a variety of methods known to those of skill including, precipitation by ammonium sulfate or sodium sulfite followed by dialysis against saline, ion exchange chromatography, affinity or immunoaffinity chromatography as well as gel filtration, zone electrophoresis, etc. (Goding in, Monoclonal Antibodies: Principles and Practice, 2d ed., pp. 104-126, Orlando, Fla., Academic Press).

### IV. Methods of Use

The methods disclosed herein are based on the discovery of celiac-specific peptides. Accordingly, in one embodiment, celiac-specific peptides can be used to detect the presence of antibodies against the peptides in a biological specimen from a subject, indicating that the subject has CD. Antibodies which bind to celiac-specific peptides can be used in a method for identifying antigens immunoreactive with anti-CSE or anti-DGP antibodies in a food sample.

A. Method for Diagnosing a Disease or Disorder

In the method for diagnosing celiac disease for example, a peptide represented by SEQ ID NO: 2, is contacted in vitro with a sample to be assayed. The step of contacting is used to enable antibodies in the sample to bind to an epitope of the peptide represented by SEQ ID NO: 2. Accordingly, this step is carried out under conditions and in an environment allowing specific antigen-antibody binding. Suitable conditions are well-known to those skilled in the art. The contacting is preferably carried out for a period of time allowing formation of a specific antigen-antibody bond between the peptide and a peptide-specific antibody possibly included in the sample.

The sample to be assayed includes any sample from a subject that contains antibodies. For example, the sample can be serum, plasma, CSF, saliva, or urine.

The celiac-specific peptide can be immobilized on a support during one or more or all steps of the procedure. For example, molecules and/or surfaces configured so as to allow reversible or irreversible binding of the peptide can be used as support. To this end, the support and/or the peptide may have functional groups which promote and/or permit binding between peptide and support. Molecules such as BSA, tTG, or surfaces such as presented by microparticles, nanoparticles or magnetic beads, or surfaces of selected membranes, polymers (e.g. polystyrene), or microtiter plates or test strips comprising such surfaces may be mentioned as exemplary supports. Suitable supports and possible ways of binding peptide and support are well-known to those skilled in the art.

The presence of the antibody bound to the solid support immobilized CSE specific peptide can be detected by any means known in the art. Preferably, the method described herein can be performed in the form of an immunoassay procedure.

In a preferred embodiment, the method is an ELISA procedure (ELISA: enzyme-linked immunosorbent assay). Direct and competitive ELISA for antibody binding to the synthetic peptides are known in the art and are described for example in Lunardi, et al., Nat Med, 6:1183-1186 (2000); Goldsby, et al., Enzyme-Linked Immunosorbent Assay; in: Immunology, 5^{th} ed., pp, 148-150. W. H. Freeman, New York, 2003. To this end, a biological sample is contacted with a celiac specific peptide described herein, immobilized on a support. Unbound components are partially or substantially removed, if necessary, and an antibody coupled or couplable to a functional group is subsequently used to detect a sample antibody bound to the peptide. As a rule, detection proceeds via a visually detectable reaction. For example, the antibody used in detection can be specific for antibodies of a particular organism or a particular origin and/or for a specific form of antibody, preferably for a particular isotype, e.g. IgA, IgM and/or IgG type antibodies, more preferably for human IgA, IgM and/or human IgG.

The method described herein can also be carried out in other assay formats, preferably e.g. as an RIA (radioimmunological assay) or as an immunoassay in a test strip format.

B. Method for Identifying Celiac Related Antigens

Anti-CSE antibodies described herein can be used in a method for identifying and eliminating from the diets of patients with CD, foods that contain antigens immunoreactive with anti-CSE antibodies. The anti-CSE antibodies can be used, for example, to the detect pathogens, e.g. peptidic pathogens, associated with celiac disease, preferably in vitro. Such pathogens can be detected in foods in order to approve certain foods for celiac patients or delete them from a list of tolerable foods. Preferably, purified antibodies or purified fragments of the antibodies including for example, Fv, F(ab′)₂. Fab fragments (Harlow and Lane, 1988, Antibody, Cold Spring Harbor Laboratory Press) are used.

### V. Kits

Kits for carrying out the methods described herein are also provided. In one embodiment, the kit is used in a method for diagnosing a subject with CD. To this end, the kit may include a CSE peptide immobilized on a support. In one embodiment, the kit includes a peptide including SEQ ID NO: 1. In a preferred embodiment, the peptide is SEQ ID NO: 2. The kit may also include agents for the detection of antibodies, preferably IgA, IgM and/or IgG type antibodies, more preferably for the detection of antibodies of human origin.

In a second embodiment, the kit is used in a method for identifying and eliminating from the diets of patients with CD, foods that contain antigens immunoreactive with anti-CSE antibodies. The kit can be used, for example, in a method to the detect pathogens, e.g. peptidic pathogens, associated with celiac disease, preferably in vitro. In this embodiment, the kit includes anti-CSE antibodies, preferably immobilized on a solid support.

In a preferred embodiment, the kit is designed in the form of an ELISA, including a secondary anti-IgG or IgA antibodies for detecting peptide binding antibodies or antibodies bound to CSE peptides.

The kit according may optionally include additional components for carrying out the methods described herein. For example, such components may include reaction vessels, filters, solutions and/or other agents. In addition, the kit may include instructions for using the kit and/or performing diagnosing a subject with CD, or detecting peptidic pathogens, associated with celiac disease.

The present invention will be understood by the following not limited examples.

### EXAMPLES

Bacterial display peptide libraries were used to first screen for disease-specific antibody binding peptides, and subsequently to evolve peptides in order to achieve diagnostically useful levels of sensitivity and specificity. Celiac disease (CD) was selected as a model disease since two distinct antibody specificities, transglutaminase 2 (TG2) and deamidated gliadin, have been characterized extensively (Kagnoff, et al., J Clin Invest 117(1):41-49 (2007)), and serve as clinically important antibody biomarkers.

### 1. Celiac Studies

Material and Methods

Reagents and strains. Reagents were supplied as follows: Dynobeads® MyOne™ Streptavidin C1 (Invitrogen), protein A/G beads (Pierce), biotinylated goat anti-human IgA, IgG, and IgM (Jackson Immunoresearch), streptavidin-R phycoerythrin conjugate, SA-PE (Invitrogen), and Quanta Lite™ Gliadin IgG II (INOVA Diagnostics) were used without modification. An Open tTG™ tissue transglutaminase (TG2) ELISA Kit (Zedira) was used with minor modifications. All cell surface experiments were performed with E. coli MC1061 (FaraΔ 139 D(ara-leu)7696 galE15 galK16 Δ (lac)X74 rpsL (StrR) hsdR2 (rK−mK+) mcrA mcrB1) (Casadaban and Cohen. JMB 1980) using the pB33eCPX surface display vector (Rice J J, PEDS, 2008).

Patient sample information and sample preparation. Serum samples were collected at the University of Tampere and Tampere University Hospital (Finland) and the Mayo Clinic, (Rochester, Minn.).

Active CD patients (n=54) ranging in age from 18-74 had small intestinal biopsies with a Marsh 3a-3c histological lesion, tested seropositive for transglutaminase (TG2) and endomysium (EMA) autoantibodies, and were Caucasian. Human lymphocyte antigens (HLA)-typing for the presence of DQ2 or DQ8 alleles was available for 42/54 CD sera. Recovering CD patients on a strict gluten-free diet (GFD) for one year (n=11) (University of Tampere) had Marsh 0-2 histological lesions, were seronegative for (anti-deamidated-gliadin (DGP) antibodies, TG2 and EMA autoantibodies, and exhibited a good overall clinical response to a GFD.

Healthy individuals derived from a volunteer population (n=50) ranging in age from 26-96 were asymptomatic for CD and tested seronegative for TG2 and EMA autoantibodies. Sera from control subjects (n=49) with various gastrointestinal (GI) illnesses were used as a discriminator cohort. Disease control subjects (18-81 yrs) were negative for CD by biopsy, and TG2 and EMA serology. These individuals exhibited Irritable Bowel Syndrome (n=16), dyspepsia (n=26), lactose intolerance (n=3), autoimmune hypothyreosis (n=1), ulcerative colitis (n=1), collagenous colitis (n=1), gastroesophageal reflux GER (n=1), and sideropenic anemia (n=1). Active CD patients from a blinded cohort (n=38) (Mayo Clinic) were similarly diagnosed by biopsy, andTG2 and EMA serology while non-CD subjects (n=40) were all seronegative (or TG2 antibodies. Serum dilutions for library screening individual patient assays ranged from 1:100 to 1:1000 in PBS-T (0.1% Tween-20) as indicated.

Depletion of E. coli binding serum antibodies. Diluted sera were depleted of E. coli binding antibodies prior to library screening and clone reactivity assays. To remove E. coli binding antibodies, an overnight culture of cells expressing the library scaffold (eCPX) with or without the v114 peptide were diluted (1:50) in fresh media and grown separately until the optical density at 600 nm (OD₆₀₀) was ˜0.6. Cells were induced for 1 hr at 37° C., and then 2×10⁹ cells from each culture were combined and centrifuged at 3000 ref for 5 min. Cells were resuspended in PBS-T (1 mL) containing 100× diluted pooled or individual sera. Samples were incubated overnight at 4° C. on an orbital shaker at 20 rpm. After incubation, samples were centrifuged at 3000 ref for 5 min (2×), and the depleted serum supernatant was recovered and stored at 4° C. for up to three days before use.

Bacterial display peptide library screening. Bacterial display peptide libraries of the form X₁₅, X₄CX₇CX₄, or X₁₃CX₂ were screened using FACS and MACS to identify peptides binding to antibodies in CD patient sera but not in non-CD sera. The library was first depleted of E. coli binding antibodies using MACS, and then depleted of non-CD (i.e., healthy and GI-illness controls) antibody binding peptides using MACS.

A frozen aliquot of each library containing 20× the expected diversity was inoculated into 500 mL LB (10 g tryptone, 5 g yeast extract, and 10 g/L NaCl) supplemented with 34 μg/mL chloramphenicol (Cm) and grown to OD₆₀₀=0.5 at 37° C. with vigorous shaking (250 rpm). Protein expression was induced by addition of L(+)-arabinose to a final concentration of 0.02% w/v with shaking at 37° C. for 1 hour. Cells (2.5×10¹⁰ cells) were centrifuged (3000 g, 4° C., 10 min) and resuspended in cold PBST.

To deplete the library of streptavidin- and protein A/G-binding clones, washed streptavidin-conjugated beads and protein A/G beads were added to a ratio of one bead per 50 cells, and the mixture was incubated 45 min at 4° C. on an inversion shaker. A magnet was then applied to the tube for 5 min and the unbound cells in the supernatant were recovered. To deplete the library of secondary antibody-binding peptides, a 1:500 dilution of secondary antibody was incubated with the cells followed by incubation with SA beads and removal by magnet similar to SA-binding peptide removal. Subtractive MACS steps for removal of non-specific serum antibody binding peptides were performed in a similar manner to that of SA & protein A/G depletions except that prior to incubation with biotinylated secondary antibody or beads, the library was first incubated with 1:100 pooled non-CD sera (n=8) for 45 min at 4° C., followed by 2× washing with PBST. For positive selection, pooled CD sera 1:100-1:200 (n=8) was added to the library and incubated for 45 min. Magnetic separation was used to wash the beads 3× with PBST, and the pellet was resuspended in LB with Cm and 0.2% glucose (w/v) for overnight growth.

For flow cytometric analysis and sorting, induced cells corresponding to 5× the estimated remaining clonal diversity were incubated with 1:100-1:200 dilution of pooled sera for 45 min at 4° C. followed by centrifugation and removal of unbound supernatant (3× washing). The pellet was then resuspended in the respective 1:500 biotinylated goat anti-human secondary antibody (IgA/IgG/IgM) in 1× PBST at 4° C. for 45 min followed by centrifugation and removal of unbound supernatant (2× washing). For tertiary labeling, the pellet resuspended in 15 nM SA-PE in 1× PBST at 4° C. for 45 min, followed by another wash via centrifugation and resuspension (3× washing) in ice-cold PBST at a volume between 10⁷ and 10⁸ cells/mL. Resuspended cells were analyzed using a FACSAria cell sorter (Becton Dickinson) using 488-nm excitation. After sorting, retained cells were amplified for further rounds of sorting by overnight growth and plated to isolate single clones.

Epitope evolution by cytometric screening. Second-generation libraries were constructed of the form X₆PEQX₆ and X₆[E/D]XFV[YF]QCX₄ (SEQ ID NO: 15) on the N-terminus of eCPX using degenerate NNS oligonucleotides (32) (shown below) resulting in an estimated library diversity of 2×10⁸ and 1×10⁸ members, respectively. A third-generation library of the form X₅PEQXFPX₄ (SEQ ID NO: 16) and X₄D[STA]FV[YF]QX₅ (SEQ ID NO: 17) was similarly constructed using oligonucleotides as shown below.

X₆PEQX₆
FW primer
(SEQ ID NO: 18)
ACTTCCGTAGCTGGCCAGTCTGGCCAG(NNS)₆CCGGAACAG(NNS)₆GG
AGGGCAGTCTGGGCAGTCTG
REV primer
(SEQ ID NO: 19)
GGCTGAAAATCTTCTCTC.
X₅PEQXFPX₄
(SEQ ID NO: 16)
FW primer
(SEQ ID NO: 20)
ACTTCCGTAGCTGGCCAGTCTGGCCAG(NNS)₅CCGGAACAGNNSTTTC
CG(NNS)₄GGAGGGCAGTCTGGGCAGTCT
REV primer
(SEQ ID NO: 21)
GGCTGAAAATCTTCTCTC.

Directed library screening was performed as above except that unique non-repeating pools of CD patient sera (n=3 subjects/pool) were used for each round of enrichment such that no pool was used no more than once and a non-repeating non-CD control pool (n=3-5 subjects/pool) was used for each round of subtraction. In effort to expand upon the known antigenic sequence, third-generation libraries were screened using 1:500 and 1:1000 pooled disease sera. PEQ focused libraries were screened by IgG isotype and DXFV^{F}/_{Y}Q (SEQ ID NO: 22) libraries using IgA isotype.

Directed evolution pooled clone & individual patient single clone reactivity assays. To compare the reactivity of consensus epitopes from each generation, overnight cultures of individual clones within a consensus epitope family (4-5 clones/family) were diluted (1:50) in fresh media and grown until the OD₆₀₀ was between 0.5 and 0.7. Cultures of each individual Clones were induced for 45 min, and 10⁶ cells from each culture were pooled together. Pooled consensus epitope families were assayed for binding to case and control group sera (3 subjects/group) by flow cytometry. In addition to MFI (B-576 channel) values, the percentage of cells from each consensus group binding to the subjects' sera was used as a measure of antibody reactivity to the displayed peptide(s). Cells expressing the eCPX scaffold without a peptide insert were used as a negative (background) control. To measure the reactivity of individual isolated mimotopes with individual patient sera, assays were similarly performed, except that a 1:100 or 1:200 serum dilutions were used for IgA and IgG isotype analysis, respectively. Non-parametric analysis tests including Wilcoxon signed rank test and Spearman's rank correlation coefficient were used to determine statistical significance and correlation values. Significance cutoff was defined as p<0.05.

TG2 and gliadin mimicry assay. To determine whether peptides sharing the DxFVYQ (SEQ ID NO: 11) consensus motif could mimic a TG2 autoantibody epitope, CD patient sera were depleted of DxFVYQ (SEQ ID NO: 11) binding antibodies using peptides containing DCFVYQC (SEQ ID NO: 14) and CxDxFVYQC (SEQ ID NO: 23) epitopes, and tested for TG2 antibody reactivity using open TG2 ELISA before and after depletion. To determine if the D_{X}FVYQ (SEQ ID NO: 11) motif could potentially mimic alpha-gliadin, individual CD patient sera was similarly depleted of DxFVYQ (SEQ ID NO: 11) binding antibodies and assayed for reactivity to an alpha-gliadin nonapeptide (PEQLPQFEE) (SEQ ID NO: 24) displayed on the N-terminus of eCPX.

Protein database queries for candidate antigen identification. The peptide insert identity of clones obtained from the library was determined by DNA sequencing and alignments and consensus motifs were generated using Geneious Pro™. Protein epitopes having high similarity to consensus motifs were identified using BLASTp searches of the UniProtKB/Swiss-Prot protein database and ranking hits by total score and E-value.

Results

Discovery of celiac disease-specific peptide epitopes. Bacterial display random peptide libraries of the form X₁₅, X₁₂CX₃ and X₄CX₇CX₄ were screened using fluorescence-activated cell sorting (FACS). For screening, individual patient sera were pooled into three groups of CD cases and three groups of non-CD sera (i.e., healthy and GI-illness control subjects) individuals, with each group composed of sera pooled from eight subjects. Alternating rounds of library enrichment were performed with CD sera using FACS and subtraction with non-CD sera using magnetic cell sorting (MACS) (FIG. 1A). To determine whether enriched library members were specific for sera from CD groups and thereby guide screening, flow cytometry was applied to quantitatively measure reactivity levels after each cycle of sorting (FIG. 1B). To systematically evaluate differences between distinct immunoglobulin classes, libraries were sorted independently based on isotype-specific reactivity using anti-IgG, anti-IgA, and anti-IgM secondary reporters. Alternating cycles of enrichment/subtraction resulted in large reactivity differences between pooled CD and non-CD sera for IgA and IgG, but not IgM, binding peptides (FIGS. 1C and 1D). Peptide sequences from IgG and IgA isotype specific library screening revealed two prevalent epitopes among 195 clones: PEQ and ^{E}/_{D}xFV^{F}/_{Y}Q (SEQ ID NO: 16) (Table 4A, Table 4B).

TABLE 4A
Sequences of individual peptides from the three most abundant
consensus groups in each cycle of epitope evolution.

| Random Peptide Library | SEQ | Focused library 1 | SEQ | Focused library 2 | SEQ |
|---|---|---|---|---|---|
| X₁₅ | ID NO | X₆PEQX₆ | ID NO | X₅PEQXFPX₄ | ID NO |
| GVGGEAHPEQTFKYDEN | SEQ ID | TLVNVRPEQPVYFGG | SEQ ID | VMEPFPEQGFPGSRA | SEQ ID |
| | NO: 285 | | NO: 299 | | NO: 314 |
| VTNFMPEQTWLGFRP | SEQ ID | RVYLGGPEQPLPVVR | SEQ ID | GPQPFPEQLFPDPFR | SEQ ID |
| | NO: 286 | | NO: 300 | | NO: 315 |
| PEQTFSGSGWH | SEQ ID | RVKLLGPEQPLHWGG | SEQ ID | AEQPFPEQGFPSGTG | SEQ ID |
| | NO: 287 | | NO: 301 | | NO: 316 |
| PGNRDAWIAHPEQRF | SEQ ID | ALVMALPEQPVPRAG | SEQ ID | SPEPFPEQGFPGIM | SEQ ID |
| | NO: 288 | | NO: 302 | | NO: 317 |
| TASGGPEQPFGSGQG | SEQ ID | VAWTMGPEQPLVRAL | SEQ ID | FRMPFPEQRFPRSGG | SEQ ID |
| | NO: 289 | | NO: 303 | | NO: 318 |
| AQAPEQARPIGSGGS | SEQ ID | GQGQAFPEQGSVPIN | SEQ ID | PNVAFPEQAFPRGAI | SEQ ID |
| | NO: 290 | | NO: 304 | | NO: 319 |
| PEQLLPQTVVGWL | SEQ ID | QPLTVFPEQEVSNRT | SEQ ID | SVEAFPEQRFPRAER | SEQ ID |
| | NO: 291 | | NO: 305 | | NO: 320 |
| TMQAAPEQVRPTDRA | SEQ ID | RQGQAFPEQVVQVSH | SEQ ID | PPWAFPEQVFPAVGL | SEQ ID |
| | NO: 292 | | NO: 306 | | NO: 321 |
| AFGPPEQVGPTYGNW | SEQ ID | QPKMSFPEQDQSVIR | SEQ ID | PTDAFPEQSFPRHLV | SEQ ID |
| | NO: 293 | | NO: 307 | | NO: 322 |
| | | QDEIAFPEQGMRWGG | SEQ ID | | |
| | | | NO: 308 | | |
| MAAVERPEQLLIEPR | SEQ ID | | | TLFMQPEQSFPERRG | SEQ ID |
| | NO: 294 | | | | NO: 323 |
| PEQLLPQTVVGW | SEQ ID | VWDRGVPEQMFPRKG | SEQ ID | GQPEQAFPEGMA | SEQ ID |
| | NO: 295 | | NO: 309 | | NO: 324 |
| RVVNMNSRPEQVVEG | SEQ ID | SVGQWLPEQMFPFA | SEQ ID | RRSEQPEQAFPDGLR | SEQ ID |
| | NO: 296 | | NO: 310 | | NO: 325 |
| RAPEQIMEFGRPWE | SEQ ID | LGGGERPEQQFPVVW | SEQ ID | LARVQPEQSFDFML | SEQ ID |
| | NO: 297 | | NO: 311 | | NO: 326 |
| GAWHTVGAPEQVQTH | SEQ ID | GGISLGPEQAWPVA | SEQ ID | RGRAQPEQAFPESVG | SEQ ID |
| | NO: 298 | | NO: 312 | | NO: 327 |
| | | RFAWLGPEQAFPIG | SEQ ID | | |
| | | | NO: 313 | | |

TABLE 4B
Sequences binding to antibodies from individuals with CD
from a constrained library of the form X₄CX₇CX₄.

| | | | | | |
|---|---|---|---|---|---|
| GQSGQPEQFMALCDCTRAEW | SEQ ID | GQSGQLRGLCPEQSGTSCRVGR | SEQ ID | GQSGQVYDSECEDSYVFQCW | SEQ ID |
| | NO: 328 | (2) | NO: 351 | | NO: 374 |
| GQSGQPEQICAIGRVGSCT | SEQ ID | GQSGQERLMCPEQAMMYCRWNN | SEQ ID | GQSGQAGRHCANTFVY?CSA | SEQ ID |
| | NO: 329 | (2) | NO: 352 | | NO: 375 |
| GQSGQPEQVCSRMRMTWCHSFG | SEQ ID | GQSGQAVGVCPEQAFDRVAHVR | SEQ ID | GQSGQTWRGCEESFVTQCPDAM | SEQ ID |
| | NO: 330 | | NO: 353 | | NO: 376 |
| GQSGQPEQICAIGRVGSCTN | SEQ ID | GQSGQHLQLCPEQDWVGCGGGR | SEQ ID | GQSGQESNTCDLFVWQACDGKQ | SEQ ID |
| | NO: 331 | | NO: 354 | (2) | NO: 377 |
| GQSGQPEQLCSSTDDAGCAYRR | SEQ ID | GQSGQYDPSCPEQMFARCLMPG | SEQ ID | GQSGQAEVACEDNFVYQCSDDW | SEQ ID |
| | NO: 332 | | NO: 355 | (6) | NO: 378 |
| GQSGQLPEQCLAHLSGQCGSKG | SEQ ID | GQSGQCPEQVLAWQQPVCKKS | SEQ ID | GQSGQSSASCDMFVYQGCAEFN | SEQ ID |
| | NO: 333 | | NO: 356 | | NO: 379 |
| GQSGQPEQICAIGRVGSCTNG | SEQ ID | GQSGQSPEQCRAWNRSPCEFMP | SEQ ID | GQSGQRQGACVDDYVYQCGHFE | SEQ ID |
| | NO: 334 | | NO: 357 | | NO: 380 |
| GQSGQPEQKCHALNDACRYIE | SEQ ID | GQSGQTPQCWPRWYGVCSVFP | SEQ ID | GQSGQGHTACMTDFVHQCFPGT | SEQ ID |
| | NO: 335 | | NO: 358 | (2) | NO: 381 |
| GQSGQPEQPCAAGMQDSCWLRS | SEQ ID | GQSGQVALWRMTWRGPEQAW | SEQ ID | GQSGQPCVDAFVYQQSGCNIA | SEQ ID |
| | NO: 336 | | NO: 359 | | NO: 382 |
| GQSGQPEQICAIGRVGSCTN | SEQ ID | GQSGQNDVPEQRWCNARCSRT | SEQ ID | GQSGQGRAACVDDFVYQCVRQHE | SEQ ID |
| | NO: 337 | | NO: 360 | | NO: 383 |
| GQSGQPEQPCAGGQRRQCLWDW | SEQ ID | GQSGQDSMPEQLWGQNMHTM | SEQ ID | GQSGQNTVVCLDGFVFQCNEWA- | SEQ ID |
| | NO: 338 | | NO: 361 | | NO: 384 |
| QSGQPEQMREWHDSDVCSMG | SEQ ID | GQSGQEGGLKSTSPEQVWGL | SEQ ID | GQSGQSQWGCMDGFWQCGGK- | SEQ ID |
| | NO: 339 | | NO: 362 | | NO: 385 |
| GQSGQPEQLCAVRQSEWCGVRW | SEQ ID | GQSGQRPPEQAWPEEVGMHS | SEQ ID | GQSGQGDTFCRDSFVYQCPRFY | SEQ ID |
| | NO: 340 | | NO: 363 | | NO: 386 |
| GQSGQTGGGCRQLPEQVCSLYY | SEQ ID | GQSGQEDPEQVWGAVPNLRV | SEQ ID | GQSGQVEDVCFDGFVFQCTG | SEQ ID |
| | NO: 341 | | NO: 364 | (2) | NO: 387 |
| GQSGQRVVNMNSRPEQVVEG | SEQ ID | GQSGQGHITMPEQEWSWGNL | SEQ ID | GQSGQALGDC-DSFVFQVCEGRD | SEQ ID |
| | NO: 342 | | NO: 365 | | NO: 388 |
| GQSGQGAWHTVGAPEQVQTH | SEQ ID | GQSGQRVGMGLHWPEQGFPE | SEQ ID | GQSGQGLRMCTDSFVNQCELWP | SEQ ID |
| | NO: 343 | | NO: 366 | | NO: 389 |
| GQSGQAFGPPEQVGPTYGNW | SEQ ID | GQSGVLPEQHWQLCRGCVRD | SEQ ID | GQSGQRDGHCADSFFVNQCVRPL | SEQ ID |
| (3) | NO: 344 | | NO: 367 | (2) | NO: 390 |
| GQSGQRAPEQIMEPGRPWER | SEQ ID | GQSGQGDTQCGMNFMTQCFPEQ | SEQ ID | GQSGQSYPSCLESFVFQCTPDW | SEQ ID |
| | NO: 345 | | NO: 368 | | NO: 391 |
| GQSGQESVPEQLCPWGVCQGR | SEQ ID | GQSGQTDDPFPDQVNETCLIM | SEQ ID | GQSGQTGRLCRESFVYQCVNKW | SEQ ID |
| | NO: 346 | | NO: 369 | | NO: 392 |
| GQSGQTGGGCRQLPEQVCSLYY | SEQ ID | GQSGQVYNDMAPDQACGVGA | SEQ ID | | |
| | NO: 347 | | NO: 370 | | |
| GQSGQESVPEQLCPWGVCQGR | SEQ ID | GQSGQEHLGADPEQRVACIGS | SEQ ID | | |
| | NO: 348 | | NO: 371 | | |
| GQSGQGAEAPEGRMHGCCAVS | SEQ ID | GQSGQANLMKPEQEMGYLKV | SEQ ID | | |
| | NO: 349 | | NO: 372 | | |
| GQSGQV-PEQTRSRGELDCCWG | SEQ ID | GQSGQRMRGCEGGPEQGCLMAH | SEQ ID | | |
| | NO: 350 | | NO: 373 | | |

Peptides with the PEQ tripeptide emerged from both linear and constrained libraries, while those with ^{E}/_{D}xFV^{F}/_{Y}Q (SEQ ID NO: 16) were identified almost exclusively from the constrained library pool.

In vitro evolution of CD specific peptides. To improve the reactivity of, and consensus between first-generation peptides, a focused library of the form X₆PEQX₆ was screened as above. Pooled sera groups (n=3 subjects/group) were used only once for library enrichment to favor peptides cross-reactive with many CD patients. The X₆PEQX₆ library was enriched for IgG and IgA specific binders, but IgG binders were more rapidly enriched and cross-reactive to multiple CD groups in comparison to IgA binders; thus, our subsequent analysis focused on IgG isotype reactivity. From the enriched library population, three highly represented consensus motifs were observed: PEQxFP (SEQ ID NO: 17), PEQPL, (SEQ NO: 18), and ^{A}_{/V}FPEQ (SEQ ID NO: 19) (Table 4A). To assess the diagnostic sensitivity and specificity of individual peptides, the reactivities of one representative clone from each motif group was measured using CD case (n=18) and non-CD control sera (n=5) not used for screening. The PEQxFP (SEQ ID NO: 21) motif derived peptide VWDRGVPEQMFPRKG (SEQ ID NO: 20) reacted with 18/18 CD sera, while VAWTMGPEQPLVRAL (SEQ ID NO: 21) to 11/18, and GQGQAFPEQGSVPIN (SEQ ID NO: 22) to 14/18. None of the peptides were reactive with control sera.

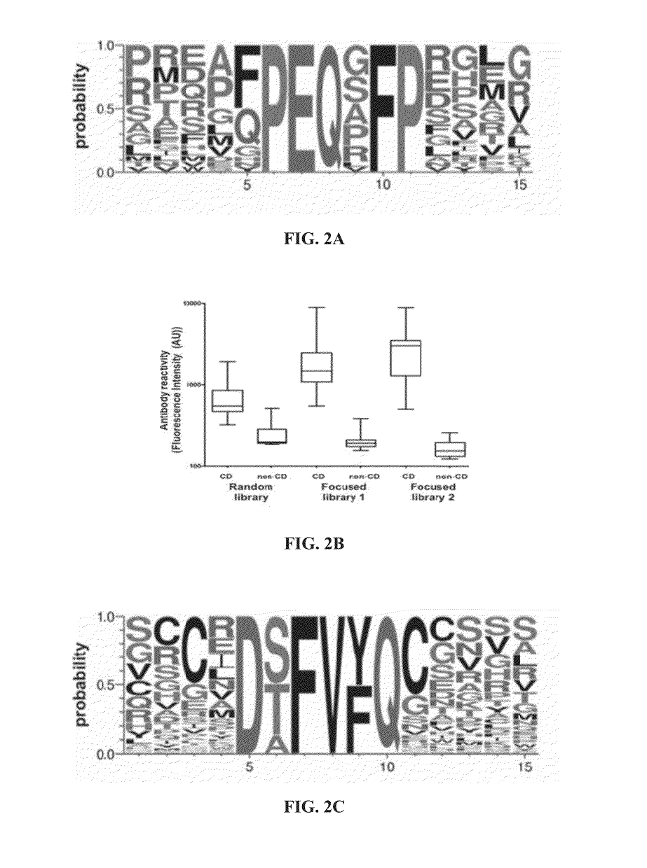

To increase the information content and diagnostic performance of the most reactive consensus motif, a second cycle of epitope expansion was performed. Thus, a library of the form x₅PEQxFPx₄ (SEQ ID NO: 16) comprised of 10⁸ members was screened as above using sera dilutions of 1:500 and 1:1000. Epitopes identified from the final screening cycle exhibited an evolved consensus of PFPEQxFP (SEQ ID NO: 6), AFPEQxFP (SEQ ID NO: 24), or QPEQ^{A}/_{S}FPE (SEQ ID NO: 25), (Table 4A, FIG. 2A), Collectively, the entire set of peptides obtained from the second focused library exhibited the evolved consensus dodecamer sequence P(^{P}/_{R}/^{M})E^{P}/_{A}^{Q}/_{F}PEQ(^{A}/_{P})FP^{E}/_{D} (SEQ ID NO: 26) (FIG. 2A), after adjusting the final position for the overrepresentation of arginine that results from NNS-codon generated RPLs. To assess whether epitope evolution improved the sensitivity and specificity of the identified peptide epitopes, four to five clones from each PEQ motif group (Table 4A) were pooled and assayed for reactivity with pooled sera from five CD or non-CD subjects. Pooled clones from each expansion cycle exhibited increased reactivity (p<0.0001) with CD patient sera, and decreased reactivity with non-CD sera (p<0.0001) (FIG. 2B), demonstrating that epitope expansion increased the diagnostic sensitivity and specificity of the identified peptides. Thus, in vitro directed evolution yielded peptides epitopes specifically recognized by CD patient IgG antibodies.

To evolve the ^{E}/_{D}xFV^{F}/_{Y}Q (SEQ ID NO: 16) epitope, a second generation library of the form X₆^{E}/_{D}xFV^{Y}/_{F}QCX₄ (SEQ ID NO: 27) was screened. This library was more readily enriched for IgA, rather than IgG binders. Additional consensus residues emerged within the randomized region and cysteine-constrained epitope variants were preferred, including CRD^{S}/_{T}FV^{F}/_{Y}-QC (SEQ ID NO: 28), RCxD^{S/}_{T}FV^{F}/_{Y}QC (SEQ ID NO: 30), and DCFV^{F}/_{Y}QC (SEQ ID NO: 31) (Table 4A, Table 5).

TABLE 5
Sequences of individual peptides from the ^{E}/_{D}XFV^{F}/_{Y}Q (SEQ ID NO: 16)
motif in each cycle of epitope expansion

| | SEQ | | SEQ | Focused library 2 | SEQ | Focused library | SEQ |
|---|---|---|---|---|---|---|---|
| Random library | ID NO | Focused library 1 | ID NO | (constrained) | ID NO | 2 (linear) | ID NO |
| GDTDCRDSFVYQCP | SEQ ID | MDVRCRDSFVYQCHVGT | SEQ ID | CIERDSFVYQACGRY | SEQ ID | GVFIDSFVYQEVMSY | SEQ ID |
| RFY | NO: 393 | | NO: 412 | | NO: 431 | | NO: 456 |
| ALGDC-DSFVFQVC | SEQ ID | GQSGCRDSFVFQCESGS | SEQ ID | VCGTDSFVFQCGNEW | SEQ ID | SASVDSFVYQGGRD | SEQ ID |
| EGRD | NO: 394 | | NO: 413 | | NO: 432 | | NO: 457 |
| SYPSCLESFVFQCT | SEQ ID | SKNDCRDSFVFQCGSRS | SEQ ID | VRCADSFVFQQCSYP | SEQ ID | VQAYDSFVFQHVGYS | SEQ ID |
| PDW | NO: 395 | | NO: 414 | | NO: 433 | | NO: 458 |
| TGRLCRESFVYQCV | SEQ ID | TVHGCRDSFVYQCESRG | SEQ ID | YSCRDTFVFQCSVVR | SEQ ID | GREADSFVYQRGGGM | SEQ ID |
| NKW | NO: 396 | | NO: 415 | | NO: 434 | | NO: 459 |
| NTVVCLDGFVFQCN | SEQ ID | RYRSCKDSFVFQCGFMP | SEQ ID | SHCNDTFVFQCASSL | SEQ ID | QSGRDSFVYQSGNGS | SEQ ID |
| EWA | NO: 397 | | NO: 416 | | NO: 435 | | NO: 460 |
| SQWGCMDGFVWQCG | SEQ ID | GVTHCKDSFVYQCISGS | SEQ ID | CRIRDAFVFQSCSRG | SEQ ID | STFDSFVFQFPSRT | SEQ ID |
| GK | NO: 398 | | NO: 417 | | NO: 436 | | NO: 461 |
| VEDVCFDGFVFQCT | SEQ ID | | | CVSRDAFVYQGCLDT | SEQ ID | HSRFDTFVFQGH | SEQ ID |
| G | NO: 399 | | | | NO: 437 | | NO: 462 |
| ESNTC-DLFVWQAC | SEQ ID | SPERCSDSFVYQCTSQR | SEQ ID | CSGSDAFVYQCHAHG | SEQ ID | RPGLDTFVYQGTTSW | SEQ ID |
| DGKQ | NO: 400 | | NO: 418 | | NO: 438 | | NO: 463 |
| AEVACEDNFVYQCS | SEQ ID | PLSRCIDSFVYQCVSSS | SEQ ID | PSCGDAFVFQCGSRM | SEQ ID | GGGLDSFVYQSTFTA | SEQ ID |
| DDW | NO: 401 | | NO: 419 | | NO: 439 | | NO: 464 |
| SSASC-DMFVYQGC | SEQ ID | LAGRCQDSFVFQCVEPT | SEQ ID | ACRLDTFVYQHGDVC | SEQ ID | SGLIDSFVYQRELKE | SEQ ID |
| AEFN | NO: 402 | | NO: 420 | | NO: 440 | | NO: 465 |
| RQGACVDDYVYQCG | SEQ ID | AGTRCLDSFVFQCVAVG | SEQ ID | VRCVDSFVFQCTKRA | SEQ ID | RIEIDSFVYQGIVGR | SEQ ID |
| HFE | NO: 403 | | NO: 421 | | NO: 441 | | NO: 466 |
| GRAACVDDFVYQCV | SEQ ID | VTDRCYDTFVFQCARGS | SEQ ID | QRCIDTFVFQCSVSA | SEQ ID | RAIADSFVYQGGDLM | SEQ ID |
| RQHE | NO: 404 | | NO: 422 | | NO: 442 | | NO: 467 |
| PCVDAFVYQQSGCN | SEQ ID | DHSRCQDTFVFQCGSRE | SEQ ID | RRCMDSFVFQCRAAS | SEQ ID | VEMSDTFVFQSL | SEQ ID |
| IA | NO: 405 | | NO: 423 | | NO: 443 | | NO: 468 |
| AGRHCANTFVYQCS | SEQ ID | | | QGCMDTFVYQCKSSL | SEQ ID | GMITDAFVYQSHTGM | SEQ ID |
| A | NO: 406 | | | | NO: 444 | | NO: 469 |
| RDGHCADSFVNQCV | SEQ ID | LVSSEADCFVYQCLSTR | SEQ ID | LGCMDSFVYQCGSFM | SEQ ID | RSRSDSFVYQESVIH | SEQ ID |
| RPL | NO: 407 | | NO: 424 | | NO: 445 | | NO: 470 |
| GLRMCTDSFVNQCE | SEQ ID | SGRSGTDCFVYQCNGVD | SEQ ID | RPCEDSFVFQCGRYT | SEQ ID | GHVPDSFVFQTFGGE | SEQ ID |
| LWP | NO: 408 | | NO: 425 | | NO: 446 | | NO: 471 |
| RDGHCADSFVNQCV | SEQ ID | SSGSETDCFVYQCGVVL | SEQ ID | HGCEDTFVYQCGNAR | SEQ ID | | |
| RPL | NO: 409 | | NO: 426 | | NO: 447 | | |
| TWRGCEESFVTQCP | SEQ ID | SSRSSTDCFVFQCVEQG | SEQ ID | SSCEDTFVYQCITPV | SEQ ID | | |
| DAM | NO: 410 | | NO: 427 | | NO: 448 | | |
| GHTACMTDFVHQCF | SEQ ID | VLPTSGDCFVYQCDLTM | SEQ ID | QCGRDSFVFQCDYV | SEQ ID | | |
| PGT | NO: 411 | | NO: 428 | | NO: 449 | | |
| | | RTGRDRDCFVFQCHLSF | SEQ ID | SCGPDSFVYQCWSPH | SEQ ID | | |
| | | | NO: 429 | | NO: 450 | | |
| | | GLAWRVDCFVYQCQRGE | SEQ ID | CHRYDTFVYQCGSTT | SEQ ID | | |
| | | | NO: 430 | | NO: 451 | | |
| | | | | QCNVDSFVYQCWPRV | SEQ ID | | |
| | | | | | NO: 452 | | |
| | | | | CLTLDTFVYQCSRSK | SEQ ID | | |
| | | | | | NO: 453 | | |
| | | | | YTCLDTFVFQNEKCA | SEQ ID | | |
| | | | | | NO: 454 | | |
| | | | | RCWMDSFVYQRCNTV | SEQ ID | | |
| | | | | | NO: 455 | | |

Similarly, screening of a linear third generation library of the form X₆D^{S}/_{T}/^{A}FV^{F}/_{Y}QX₄ (SEQ ID NO: 32) identified a preference for cyclic peptides having the consensus CEDSFV^{F}/_{Y}QC (SEQ ID NO: 33) (FIG. 2C), and non-constrained linear epitopes with the consensus ΩD^{S}/_{T}FV^{F}/_{Y}Q (SEQ ID NO: 34), where Ω=[L/I/M/F/E] (Table 5).

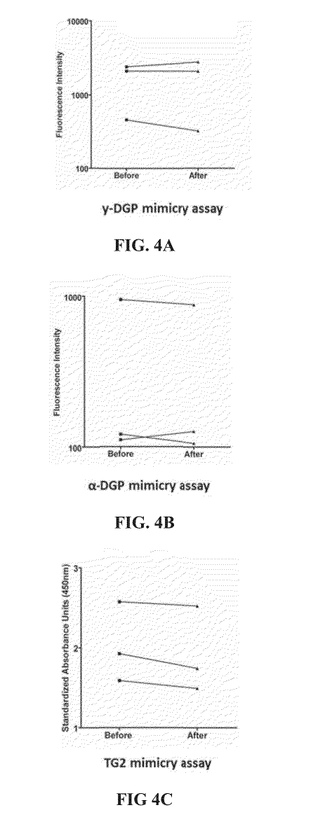

Importantly, the novel celiac specific epitope (CSE) was not a mimic of TG2 or DGP since antibody titers against these CD antigens were unaffected by depletion of antibodies binding to the novel epitope (FIG. 4A-C). Given the weak consensus at the Ω position, the degenerate search motif D^{S}/_{T}FV^{F}/_{Y}Q (SEQ ID NO: 35) was used along with ScanProsite to identify 35 candidate antigens. (Table 6)

TABLE 6

| | | AA | |
|---|---|---|---|
| Antigen candidate | Sequence | position | SEQ ID NO |
| Singapore isolate 9 (sub-type 7) whole | DSFVYQ | 242-247 | SEQ ID NO: 3 |
| genome shotgun sequence assembly, | | | |
| scaffold_4. Blastocystis hominis | | | |
| Rs element Vgr family protein 2. | DSFVYQ | 123-128 | SEQ ID NO: 3 |
| Achromobacter arsenitoxydans SY8 | | | |
| Putative uncharacterized protein. Giardia | DTFVFQ | 204-209 | SEQ ID NO: 472 |
| intestinalis (strain A TCC 50803/WB clone | | | |
| C6) (Giardia Lamblia) | | | |
| Extracellular ligand-binding receptor. | DTFVYQ | 360-365 | SEQ ID NO: 473 |
| Ilyobacter polytropus (strain DSM 2926/ | | | |
| CuHBul) | | | |
| Aminopeptidase C. Lactobacillus helveticus | DSFVYQ | 405-410 | SEQ ID NO: 3 |
| (Lactobacillus suntoryeus) | | | |
| Hypothetical lipoprotein, Leptospira biflexa | DSFVYQ | 113-118 | SEQ ID NO: 3 |
| serovar Patoc (strain Patoc 1/Ames) | | | |
| Putative xylanase/chitin deacetylase. | DSFVYQ | 385-390 | SEQ ID NO: 3 |
| Slackia exigua ATCC 700122 | | | |
| Arylsulfatase. Parasutterrella | DSFVYQ | 92-97 | SEQ ID NO: 3 |
| excrementihominis Y1T 11859 | | | |
| Putative uncharacterized protein | DTFVYQ | 182-187 | SEQ ID NO: 473 |
| B3E4.220. Neurospora crassa | | | |
| Uncharacterized protein. Tetrahymena | DSFVYQ | 73-78 | SEQ ID NO: 3 |
| thermophila (strainSB210) | | | |
| Hemolysin-type calcium-binding region | DSFVYQ | 526-531 | SEQ ID NO: 3 |
| protein. Pseudomonas fluorescens | | | |
| (strainPf0-1) | | | |
| DNA-directed RNA polymerase. | DTFVYQ | 1296-1301 | SEQ ID NO: 473 |
| Mycoplasma sp. 1220 | | | |
| Putative uncharacterized protein. | DTFVFQ | 301-306 | SEQ ID NO: 472 |
| Bacteroides plebeius (strain DSM 17135/ | | | |
| JCM/112973/M2) | | | |
| EF-hand calcium-binding domain-containing | DTFVYQ | 327-332 | SEQ ID NO: 473 |
| protein 6. Homo sapiens (Human) | | | |
| Uncharacterized protein. Puccinia triticina | DTFVYQ | 1753-1758 | SEQ ID NO: 473 |
| (isolate 1-1/race 1 (BBBD)) (Brown leaf | | | |
| rust fungus) | | | |
| Aminoglycoside phosphotransferase. | DTFVYQ | 371-376 | SEQ ID NO: 473 |
| Eggerthella lenta (strain ATCC 25559/DSM | | | |
| 2243/JCM 9979/NCTC 11813/VPI 0255) | | | |
| (Eubacterium lentum) | | | |
| Ser/Thr protein phosphatase family | DSFVYQ | 129-134 | SEQ ID NO: 3 |
| protein. Neisseria sicca ATCC 29256 | | | |
| Capsule synthesis protein, CapA. | DTFVYQ | 315-320 | SEQ ID NO: 473 |
| Geobacillus sp. (strain Y412MC61) | | | |
| Periplasmic nitrate reductase, electron | DSFVYQ | 62-67 | SEQ ID NO: 3 |
| transfer subunit. Vibrio sp. (strain Ex25) | | | |
| Modification methylase DdeI. Prevotella | DSFVFQ | 578-583 | SEQ ID NO: 474 |
| capri DSM 18205 | | | |
| Acetyl esterase. Lactobacillus | DTFVFQ | 193-198 | SEQ ID NO: 472 |
| kefiranafaciens (strain ZW3) | | | |
| D-arabinono-1,4-lactone oxidase, | DSFVYQ | 263-268 | SEQ ID NO: 3 |
| Schizosaccharomyces pombe (strain 972/ | | | |
| ATCC 24843) (Fission yeast) | | | |
| Chaperone protein DnaK. Roseburia | DSFVYQ | 513-518 | SEQ ID NO: 3 |
| inulinivprans DSM 16841 | | | |
| Uncharacterized protein. Bartonella | DSFVYQ | 172-177 | SEQ ID NO: 3 |
| vinsonii subsp. arupensis Pm136co | | | |
| Conserved hypothetical signal peptide | DSFVYQ | 508-513 | SEQ ID NO: 3 |
| protein. Burkholderia cepacia GG4 | | | |
| Major facilitator family protein. Dialister | DSFVYQ | 314-319 | SEQ ID NO: 3 |
| Invisus DSM 15470 | | | |
| Polyprotein. Leek yellow stripe virus | DTFVYQ | 67-72 | SEQ ID NO: 473 |
| Proteophosphoglycan 5. Rhodotorula | DTFVYQ | 1716-1721 | SEQ ID NO: 473 |
| glutinis (strain ATCC 204091/HP 30/ | | | |
| MTCC 1151) (Yeast) | | | |
| Protease, HINT family. Leptospira | DSFVFQ | 69-74 | SEQ ID NO: 473 |
| interrogans str. FPW2026 | | | |
| Inner-membrane translocator. Rubrivivax | DSFVYQ | 115-120 | SEQ ID NO: 3 |
| benzoatilyticus JA2 = ATCC BAA-35 | | | |
| ABC transporter permease protein. | DTFVYQ | 50-55 | SEQ ID NO: 473 |
| Bacillus cereus (strain G9842) | | | |
| 4-diphosphocytidyl-2-C-methyl-D- | DCFVYQ | 265-270 | SEQ ID NO: 475 |
| erythritol kinase. Prevotella tannerac ATCC | | | |
| 51259 | | | |
| Rhs element Vgr protein. Ralstonia sp. | DCFVYQ | 125-130 | SEQ ID NO: 475 |
| 5_7_47FAA | | | |
| Putative uncharacterized protein. | DCFVYQ | 181-186 | SEQ ID NO: 475 |
| Lachnospiraceae bacterium | | | |
| 3_1_57FAA_CT1 | | | |
| 4_diphosphocytitlyl-2-C-methyl-D- | DCFVYQ | 91-96 | SEQ ID NO: 475 |
| erythritolkinase gut metagenome | | | |

### Evolved Peptide Epitopes Exhibit High Diagnostic Sensitivity and Specificity.

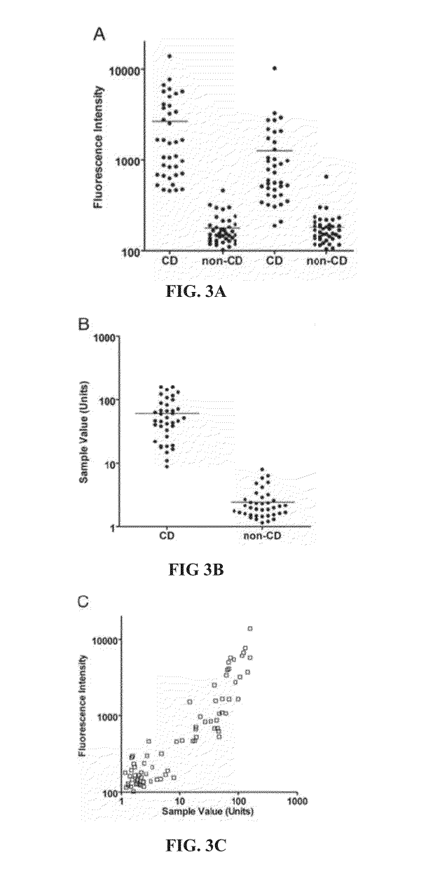

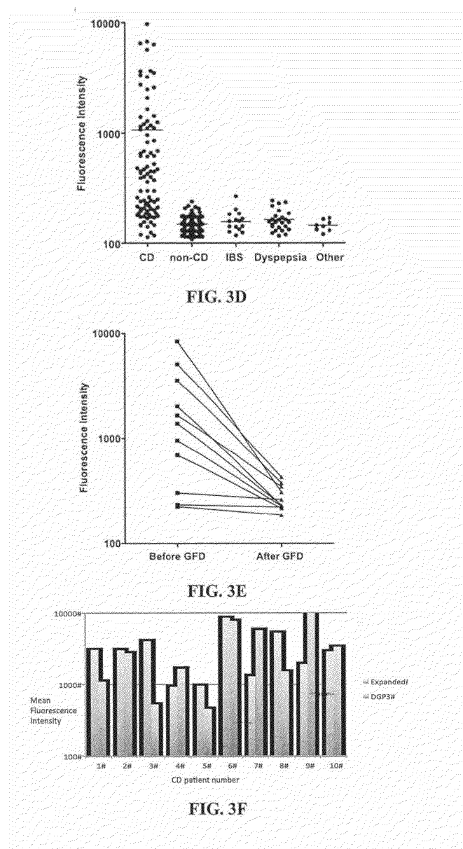

To evaluate the diagnostic utility of expanded peptide epitopes, sera from CD cases and controls (n=78) were assayed in a one-way blinded test. Two peptides newly identified peptides (DGP3: RGRAQPEQAFPESVG (SEQ ID NO: 9), DGP6: GPQPFPEQLFPDPFR (SEQ ID NO: 36)) exhibiting high sensitivity and specificity in a preliminary set of 10 CD and 10 non-CD sera, were assayed for IgG reactivity, and a diagnostic cutoff was established using the individual patient reactivity data set. Epitope DGP3 correctly identified 100% of CD cases (38/38) and 97.5% (39/40) non-CD controls; epitope DGP6 correctly identified 92.1% of CD cases (35/38) and 97.5% (39/40) non-CD controls (FIG. 3A).

For comparison, a commercially available assay Quanta Lite™ DGP IgG assay, using a cutoff value of 10 Units, achieved 98% sensitivity and 100% specificity (FIG. 3B). Furthermore, assay results with epitope DGP3 correlated with those obtained using Quanta Lite™ (FIG. 3C). Thus, a single peptide generated using sequential epitope expansion performed equivalently to a proprietary, FDA approved diagnostic assay.

To determine prevalence of anti-CSE antibodies in CD and control subjects, CD and non-CD sera (n=231) were assessed for reactivity to the CSE peptide: MDVRCRDSFVYQCHVGT (SEQ ID NO: 2). Overall, the CSE peptide exhibited 71% (65/92) sensitivity and 99% (2/139) specificity (FIG. 3D). To determine if the serum antibody titer against the CSE epitope dissipated after the introduction of a gluten-free diet (GFD), sera from 11 CD cases obtained at time-of-diagnosis or after one year on a GFD were assayed. Active CD patients (10/11) were reactive and 8/11 of these patients exhibited reduced, but non-zero, levels of epitope reactivity after a GFD (FIG. 3E); all patients were seronegative for TG2 and DGP antibodies after a GFD. Together, these results suggest the CD-specific peptide is derived from an antigen distinct from TG2 and DGP epitopes.

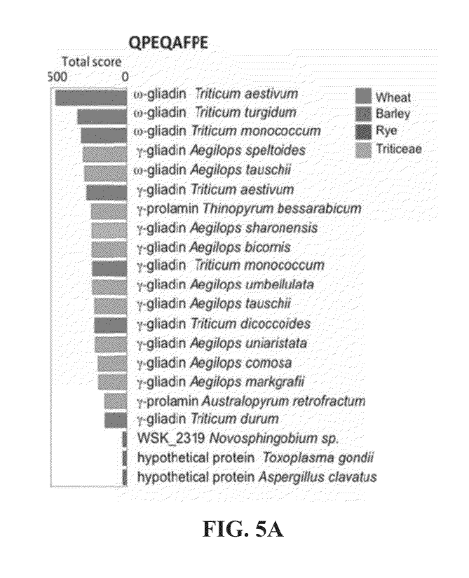

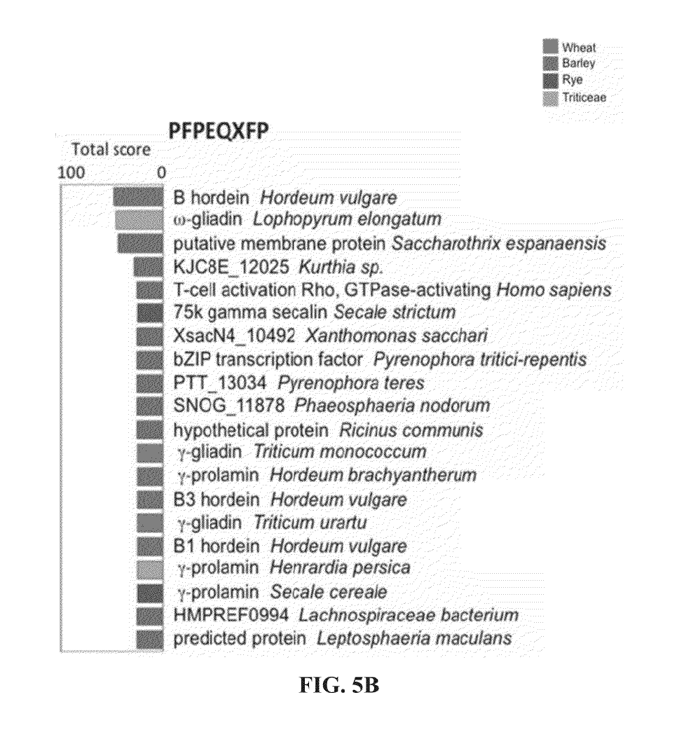

Directed evolution of peptide epitopes facilitates non-self antigen discovery. Due to the substantially increased information content within the third generation evolved consensus epitopes (QPEQAFPE (SEQ ID NO: 4), PFPEQxFP (SEQ ID NO: 6) as compared to the first generation epitope PEQ, the evolved epitopes were employed in an unbiased antigen identification within the entire protein database. Unbiased BLASTp searches of the epitopes QPEQAFPE (SEQ ID NO: 4) and PFPEQxFP (SEQ ID NO: 6) directly identified cereal grain proteins from the genus Triticeae including gliadins, hordeins, and secalins (FIGS. 5A and B). For comparison, an identical search using the first and second generation motifs PEQ (SEQ ID NO: 4) and PEQxFP (SEQ ID NO: 17) yielded an excessive number of unrelated hits, and did not enable antigen discovery. The highest scoring antigen, obtained using the epitope consensus QPEQAFPE (SEQ ID NO: 4) was ω-gliadin from wheat (FIG. 5A). Similarly, use of the aggregate (i.e., using all sequences) consensus epitope from third generation peptides (P(^{R}/_{M}/^{P})EP^{Q}/_{F}PEQ(^{A}/_{P})FPE (SEQ ID NO: 37); Table 4A) identified exclusively ω-gliadins among the 25 highest scoring sequences. Searches performed with the third-generation motif PFPEQxFP (SEQ ID NO: 6) also identified a diverse group of prolamins from wheat, barley, and rye (FIG. 5B). The third-generation motifs were identical to the prolamin epitopes that, in CD, result from post-translational deamidation of glutamine to glutamic acid (Q→E) by TG2. Collectively, these results demonstrate that the in vitro directed evolution of epitopes can facilitate discovery of non-self antigens.

Discussion

The ADEPt method presented here provided an effective route to evolve diagnostically efficacious peptides for de novo biomarker discovery and detection without knowledge of disease pathobiology. Previous methods to discover peptides binding to disease antibodies, including antibody profiling and signature analysis using peptide libraries (Cortese, et al., Trends Biotechnol. 12(7):262-267 (1994); Restrepo, et al., J. Neuroimmunol, 254(1-2):154-160 (2013)), have demonstrated the existence of unique antibody specificities in a broad range of diseases (Fierabracci, et al., Immunol Lett 124(1):35-43 (2009)). And although the peptides identified have demonstrated diagnostic potential, alone or in panel format (Kouzmitcheva, et al., Clin Diagn Lab Immunol 8(1):150-160 (2001); Fierabracci, et al., Immunol. Lett. 124(1)35-43 (2009)), their translation to the clinic has been hindered by inadequate diagnostic sensitivity and specificity values. By applying concepts from in vitro directed evolution to human patient samples, screen large libraries were screened in an iterative fashion for molecular properties (affinity and cross reactivity, and molecular specificity) that favor diagnostic sensitivity and specificity. In agreement with many prior studies, our results demonstrate that a RPL, in the absence of directed evolution, is insufficient to identify peptides with optimal diagnostic efficacy. Only when the peptide search space was expanded through directed evolution were accuracies comparable to gold-standard diagnostics for CD achieved. Thus, it may be possible to improve the diagnostic utility of previously reported peptides arising from RPLs using ADEPt. Although the directed evolution process was concluded after screening the third generation focused epitope library wherein sensitivity and specificity were maximized (100%, 98%), further cycles of directed evolution could enhance the dynamic range between CD and non-CD signals. Thus, the results demonstrate the broad utility of directed evolution in the context of biomarker discovery and diagnostics development.

Here, environmental (i.e., non-human) protein antigens recognized by CD-specific antibodies were unambiguously identified using ADEPt. Multiple methods have been developed to identify candidate autoantigens, including synthetic peptide and peptide arrays (Reddy, et al., Cell 144(1):132-142, 2011)), whole protein antigen arrays (Anderson, J. Proteome Res. 10(1):85-96 (2011), and human cDNA or peptidome libraries (Larman, 29(6):535-541 (2011)). In contrast, methods to identify non-human antigens mostly closely associated with disease have not been reported. The rapidly expanding protein database, currently composed of more than 31 million protein sequences, is simply too large to enable database searching using the limited consensus data arising from a first-generation RPL. Epitope expansion using ADEPt dramatically reduced the frequency of antigen candidates within the non-redundant protein database, enabling precise identification of immunodominant B-cell epitopes (ω-gliadin, γ-gliadin, and B-hordein). Interestingly, the immunodominant B-cell epitopes were highly similar to recently elucidated immunodominant T-cell epitopes (Tye-Din, et al., Sci. Transl. Med. 2(41):41ra51 (2010)). Linear B-cell epitopes derived from the CD-specific autoantigen TG2 were not observed, which is consistent with the proposed existence of immundominant structural epitopes within TG2 (Simon-Vecsei, et al., Proc. Acad. Sci. USA 109(2):431-436 (2012)). However, there is a possibility that lower abundance linear epitopes or structural mimotopes were enriched during library screening but outcompeted by DGP and D^{S}/_{T}FV^{Y}/_{F}Q (SEQ NO: 39) peptides. Future efforts using next-generation sequencing and bioinformatics tools may permit identification and characterization of a greater number and variety of disease-associated peptide epitopes.

Application of ADEPt to sera from CD patients identified another previously unreported celiac specific epitope (CSE). Antibodies binding CSE peptides with the consensus motif CXD^{S}/_{T}FV^{Y}/_{F}QC (SEQ ID NO: 1) were present in 71% of CD subjects from geographically distinct cohorts and exhibited equivalent specificity (˜99%) for CD when compared to gold-standard antibody biomarkers of CD (anti-TG2 IgA, anti-endomysial antibodies, and anti-DGP IgG). The sensitivity and specificity values observed with CSE are significant since many distinct antibodies have reported to be present in patients with CD but the same specificities have been observed in unrelated disorders (Alaedini, et al., Autoimmunity 41(1):)9-26 (2012); D'Angelo, et al., Clin. Immunol. 148(1):99-109 (2013)); to our knowledge, none have demonstrated diagnostic specificity values comparable to that of CSE (Alaedini, et al., Autoimmunity 41(1):19-26 (2012)). The observation that anti-CSE antibody titers significantly decrease in matched sera from patients pre- and one year post-GFD further supports the disease specificity of this antibody-specificity. Although the precise identity of the antigen mimicked by CSE remains to be elucidated, the ability of the evolved consensus epitope to narrow our search to <40 candidate antigens suggests that antigen discovery will be possible. Finally, the well-established significance of DGP antibodies and TG2 autoantibodies to the pathobiology of CD suggests that confirmation of the antigen corresponding to CSE may provide additional clues regarding the mechanisms of CD pathogenesis.

In summary, the ADEPt method disclosed herein enables the simultaneous discovery of antibody biomarkers of disease and reagents for their sensitive and specific detection. In principle, ADEPt could be applied to a variety of antibody-containing specimens (e.g., serum/plasma, CSF, urine, saliva). Given the ubiquitous nature of antibody repertoire changes observed in diverse diseases, ADEPt may be useful to create diagnostics for early disease detection, stratification, and therapeutic monitoring (Roep, et al., Nat. Med. 18(1)48-53 (2012)). Additionally, ADEPt may be useful to reveal previously unknown environmental factors involved in disease.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims

We claim:

1. A process for identifying peptides that bind antibodies from a subject comprising the steps of:
(a) contacting a cell displayed peptide library with a diluted fluid specimen from one or more subjects with disease to capture disease antibodies on the cell surface-containing sample,
(b) contacting the cell antibody-containing sample in step (a) with one or more antibody binding reporter proteins;
(c) measuring antibody binding to cells, and recovering cells that bind to antibodies in the fluid specimen;
(d) contacting the recovered cells in step (c) with diluted fluid specimens from one or more control subjects without disease;
(e) recovering cells that do not bind to control subject antibodies; and
(f) identifying one or more consensus sequences among peptides binding to disease subject antibodies but not to antibodies from subjects without disease.

2. The process of claim 1, further comprising
(g) constructing a second peptide library encompassing the consensus sequence identified from the random peptide library to form a first focused peptide library, wherein one or more sequence positions are fixed to amino acids within the consensus sequence or biased toward amino acids within the consensus sequence;
(h) contacting the first focused peptide library with a diluted fluid specimen from one or more subjects with disease to form a second antibody-containing sample;
(i) contacting the second antibody-containing sample with an antibody binding protein reporter;
(j) measuring antibody binding to cells, and recovering cells that bind to antibodies in the fluid specimen;
(k) contacting the recovered cells in step (j) with diluted fluid specimens from one or more control subjects without disease;
(l) recovering cells that do not bind to the control subjects' antibodies; and
(m) identifying one or more consensus sequences among displayed peptides binding to disease subject antibodies but not to antibodies from subjects without disease.

3. The process of claim 2, wherein the reporter protein is biotinylated anti-IgG, IgA and IgM.

4. The process of claim 1, wherein the diluted fluid specimen is selected from the group consisting of serum, plasma, CSF, saliva and urine.

5. The process of claim 1, wherein the diluted fluid specimen is from pooled from 2 to 10 subjects.

6. The process of claim 5, wherein the fluid specimen is from 2 to 5 subjects.

7. The process of claim 1, wherein the fluid specimen is diluted to a ratio from 1:100 to 1:200.

8. The process of claim 2, wherein the fluid specimen is diluted at a ratio from 1:500 to 1:1000.

9. The process of claim 1, wherein the peptide library is displayed on a bacterial cell.

10. The process of claim 2, further comprising constructing a second focused peptide library encompassing the consensus sequences identified from the first focused peptide library, wherein one or more sequence positions are fixed to amino acids within the consensus sequence or biased toward amino acids within the consensus sequence;
(m) repeating steps (h) to (k), and
(n) identifying one or more consensus sequences (referred to as “second consensus sequences”) among peptides binding to disease subject antibodies but not to antibodies from subjects without disease, wherein the disease and control subjects are different from subjects used to identify the first focused library.

11. A peptide comprising an amino acid sequence selected from the group consisting of RGRAQPEQAFPESVG (SEQ ID NO:9), PPEPQPEQAFPE (SEQ ID NO:6), PREPQPEQAFPE (SEQ ID NO:7) and PMEPQPEQPFPE, (SEQ ID NO:8).

12. The peptide according to 11, comprising the amino acid sequence of SEQ ID NO:1.

13. A method for diagnosing a subject with Celiac disease comprising:
obtaining a sample from the subject;
contacting the peptide according to claim 10 with the sample and antibody bound to the peptide.

14. The method according to claims 13, wherein the method is carried out in an ELISA format.

15. A kit for performing a method according to claim 13, comprising an amino acid sequence selected from the group consisting of SEQ ID NOs:2, 5 and 6.

16. The kit according to claim 15, wherein the kit is an ELISA kit, and wherein the kit further comprises agents for the detection of IgA and/or IgG type antibodies.

17. A peptide comprising the sequence DxFVYQ (SEQ ID NO:11) or DxFVFQ (SEQ ID NO:12).

18. The peptide of claim 17, wherein the peptide comprises a sequence selected from the group consisting of IDxFVYQGA (SEQ ID NO:13), where x is any amino acid.

19. The peptide of claim 17, wherein the peptide comprises the sequence selected from the group consisting of MDVRCRDSFVYQCHVGT (SEQ ID NO:2) and QRCIDTFVFQCSVSA (SEQ ID NO:38).

20. A fusion peptide containing the peptide QNGIDMFVYQGALA (SEQ ID NO:39) or an equivalent peptide substituted with one or more conservative amino acid substitutions.
